SEQUENCE LISTING (18 sequences)

SEQ ID NO: 1 - DNA, 2723 bp, Homo sapiens
aaaatgtgttctctaaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataaaaaaaaaaaaaaaaa

SEQ ID NO: 2 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 3 - DNA, 1227 bp, Homo sapiens
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 4 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 5 - DNA, 813 bp, Homo sapiens
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 6 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 7 - DNA, 667 bp, Homo sapiens
agcacaggacacagctgggttctgaagcttctgagttctgcagcctcacctctgagaaaacctcttttccaccaataccatgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattaggtgtcatttccattatttatattagtttagccaaaggataagtgtcccctatggggatggtccactgtcactgtttctctgctgttgcaaatacatggataacacatttgattctgtgtgttttcataataaaactttaaaataaaatgcagacagtt

SEQ ID NO: 8 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 9 - DNA, 3686 bp, Homo sapiens
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataaaaaaaaaaaaaaaaaaaaaaaaaaa

SEQ ID NO: 10 - protein, 352 aa, Homo sapiens [sequence omitted]

SEQ ID NO: 11 - DNA, 3338 bp, Homo sapiens
ataaaaagccacagatctctggcggcggcaaggggacagcactgagctctgccgcctggctctagccgcctgcctggcccccgccgggactcttgcccaccctcagccatggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtgtgaactcctctggcctgtgtctagttgtttgattcagacagctgcctgggatccctcatcctcatacccacccccacccaagggcctggcctgagctgggatgattggaggggggaggtgggatcctccaggtgcacaagctccaagctcccaggcattccccaggaggccagccttgaccattctccacctgccagggacagagggggtggcctcccaactcaccccagccccaaaactctcctctgctgctggctggttagaggttccctttgacgccatcccagccccaatgaacaattatttattaaatgcccagccccttctgacccatgctgccctgtgagtactacagtcctcccatctcacacatgagcatcaggccaggccctctgcccactccctgcaacctgattgtgtctcttggtcctgctgcagttgccagtcaccccggccacctgcggtgctatctcccccagccccatcctctgtacagagcccacgcccccactggtgacatgtcttttcttgcatgaggctagtgtggtgtttcctggcactgcttccagtgaggctctgcccttggttaggcattgtgggaaggggagataagggtatctggtgactttcctctttggtctacactgtgctgagtctgaaggctgggttctgatcctagttccaccatcaagccaccaacatactcccatctgtgaaaggaaagagggaggtaaggaatacctgtccccctgacaacactcattgacctgaggcccttctctccagcccctggatgcagcctcacagtccttaccagcagagcaccttagacagtccctgccaatggactaacttgtctttggaccctgaggcccagagggcctgcaagggagtgagttgatagcacagaccctgccctgtgggcccccaaatggaaatgggcagagcagagaccatccctgaaggccccgcccaggcttagtcactgagacagcccgggctctgcctcccatcacccgctaagagggagggagggctccagacacatgtccaagaagcccaggaaaggctccaggagcagccacattcctgatgcttcttcagagactcctgcaggcagccaggccacaagacccttgtggtcccaccccacacacgccagattctttcctgaggctgggctcccttcccacctctctcactccttgaaaacactgttctctgccctccaagaccttctccttcacctttgtccccaccgcagacaggaccagggatttccatgatgttttccatgagtcccctgtttgtttctgaaagggacgctacccgggaagggggctgggacatgggaaaggggaagttgtaggcataaagtcaggggttcccttttttggctgctgaaggctcgagcatgcctggatggggctgcaccggctggcctggcccctcagggtccctggtggcagctcacctctcccttggattgtccccgacccttgccgtctacctgaggggcctcttatgggctgggttctacccaggtgctaggaacactccttcacagatgggtgcttggaggaaggaaacccagctctggtccatagagagcaagacgctgtgctgccctgcccacctggcctctgcactcccctgctgggtgtggcgcagcatattcaggaagctcagggcctggctcaggtggggtcactctggcagctcagagagggtgggagtgggcccaatgcactttgttctggctcttccaggctgggagagccttccaggggtgggacaccctgtgatggggccctgcctcctttgtgaggaagccgctggggccagttggtcccccttccatggactttgttagtttctccaagcaggacatggacaaggatgatctaggaagactttggaaagagtaggaagactttggaaagacttttccaaccctcatcaccaacgtctgtgccattttgtattttactaataaaatttaaaagtcttgtgaatcaaaaaaaaaaaaaaaaaaaaaa

SEQ ID NO: 12 - protein, 397 aa, Homo sapiens
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 13 - DNA, 3249 bp, Homo sapiens
caagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgccaaa

SEQ ID NO: 14 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 15 - DNA, 3220 bp, Homo sapiens
aaacttttttccctggctctgccctgggtttccccttgaagggatttccctccgcctctgcaacaagaccctttataaagcacagactttctatttcactccgcggtatctgcatcgggcctcactggcttcaggagctgaataccctcccaggcacacacaggtgggacacaaataagggttttggaaccactattttctcatcacgacagcaacttaaaatgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagagaaaccatttactgttgagatctcccctggaccccggattgctgctcagattggagactcagtcatgttgacatgtagtgtcatgggctgtgaatccccatctttctcctggagaacccagatagacagccctctgagcgggaaggtgaggagtgaggggaccaattccacgctgaccctgagccctgtgagttttgagaacgaacactcttatctgtgcacagtgacttgtggacataagaaactggaaaagggaatccaggtggagctctactcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacacttgatgttcaaggaagagaaaacaacaaagactatttttctcctgagcttctcgtgctctattttgcatcctccttaataatacctgccattggaatgataatttactttgcaagaaaagccaacatgaaggggtcatatagtcttgtagaagcacagaagtcaaaagtgtagctaatgcttgatatgttcaactggagacactatttatctgtgcaaatccttgatactgctcatcattccttgagaaaaacaatgagctgagaggcagacttccctgaatgtattgaacttggaaagaaatgcccatctatgtcccttgctgtgagcaagaagtcaaagtaaaacttgctgcctgaagaacagtaactgccatcaagatgagagaactggaggagttccttgatctgtatatacaataacataatttgtacatatgtaaaataaaattatgccatagcaagattgcttaaaatagcaacactctatatttagattgttaaaataactagtgttgcttggactattataatttaatgcatgttaggaaaatttcacattaatatttgctgacagctgacctttgtcatctttcttctattttattccctttcacaaaattttattcctatatagtttattgacaataatttcaggttttgtaaagatgccgggttttatatttttatagacaaataataagcaaagggagcactgggttgactttcaggtactaaatacctcaacctatggtataatggttgactgggtttctctgtatagtactggcatggtacggagatgtttcacgaagtttgttcatcagactcctgtgcaactttcccaatgtggcctaaaaatgcaacttctttttattttcttttgtaaatgtttaggtttttttgtatagtaaagtgataatttctggaattagaaaaaaaaaaaaaaaaaaa

SEQ ID NO: 16 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 17 - DNA, 5346 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 18 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

(19) United States
(12) Patent Application Publication
(10) Pub. No.: US 2020/0123621 A1
(43) Pub. Date: Apr. 23, 2020

(54) COMPOSITIONS AND METHODS FOR IDENTIFYING AND TREATING RESISTANCE TO CTLA4 ANTAGONISTS IN LEUKEMIA

(71) Applicant: DANA-FARBER CANCER INSTITUTE, INC., Boston, MA (US)

(72) Inventor: Pavan Bachireddy, Boston, MA (US)

(73) Assignee: DANA-FARBER CANCER INSTITUTE, INC., Boston, MA (US)

(21) Appl. No.: 16/626,137

(22) PCT Filed: Jun. 26, 2018
(86) PCT No.: PCT/US2018/039446

§ 371 Date: Dec. 23, 2019

Related U.S. Application Data
(60) Provisional application No. 62/525,401, filed on Jun. 27, 2017.

(51) Int. Cl. C12Q 1/6886; C07K 16/28; A61P 35/00

(52) U.S. Cl. CPC C12Q 1/6886; C07K 16/2818; C12Q 2600/158; C07K 2317/24; C12Q 2600/106; A61P 35/00

(57) ABSTRACT

The present invention relates to compositions and methods for identifying and treating resistance to CTLA4 antagonists in neoplasia. In particular, the invention relates to compositions and methods for use in identifying and treating Ipilimumab resistant forms of leukemia.

### RELATED APPLICATIONS

This application claims the benefit of priority under 35 U.S.C. § 119(e) to U.S. Provisional Application No: 62/525,401, filed Jun. 27, 2017, which is incorporated herein by reference in its entirety.

### FIELD OF THE INVENTION

The invention relates to compositions and methods for identifying and treating resistance to CTLA4 antagonists in neoplasia. More particularly, the invention relates to compositions and methods for use in identifying and treating Ipilimumab-resistant forms of leukemia.

### BACKGROUND OF THE INVENTION

Cytotoxic T-lymphocyte-associated protein 4 (CTLA4) antagonists (e.g., Ipilimumab) can induce durable tumor remissions for some types of neoplasia (e.g., leukemia). Unfortunately, many patients with leukemia do not respond to treatment with Ipilimumab. Accordingly, prior to the invention described herein, there was an urgent need to identify more effective methods for predicting response or resistance to CTLA4 blockade.

### SUMMARY OF THE INVENTION

The invention provides techniques for the identification of gene expression patterns that discriminate the clinical outcomes of CTLA4 antagonists in neoplasia (e.g., leukemia). In particular, the techniques herein provide gene expression patterns/signatures that identify forms of leukemia that may be resistant to treatment with CTLA4 antagonists such as, for example, Ipilimumab. Prior to the invention described herein, the skilled artisan was not aware of any molecular signatures capable of precisely predicting response and resistance to CTLA4 antagonists.

In one aspect, the present disclosure provides a method of determining whether treatment of a subject having a leukemia with a cytotoxic T-lymphocyte-associated protein 4 (CTLA4) antagonist will result in clinical benefit to the subject. The method may include the following steps: obtaining a test sample from the subject having the leukemia; determining the expression level of at least one leukemia-associated gene in the test sample; comparing the expression level of the leukemia-associated gene in the test sample with the expression level of the leukemia-associated gene in a reference sample; and determining whether the CTLA4 antagonist will inhibit leukemia in the subject if the expression level of the leukemia-associated gene in the test sample is differentially expressed relative to the level of the leukemia-associated gene in the reference sample.

In an illustrative embodiment, the test sample may be obtained from a leukemia tissue, a tumor microenvironment, or a tumor-infiltrating immune cell.

In an illustrative embodiment, the clinical benefit in the subject may be a complete or partial response as defined by response evaluation criteria in solid tumors (RECIST), stable disease as defined by RECIST, or long-term survival in spite of disease progression or response as defined by irRC criteria.

In an illustrative embodiment, the test sample may be obtained from the leukemia and the leukemia-associated gene may be a CD47 molecule (CD47) gene. In this case, if the expression level of the CD47 gene in the test sample is higher than the level of the CD47 gene in the reference sample, then treatment of the subject with leukemia with the CTLA4 antagonist will not result in clinical benefit in the subject. However, if the expression level of the CD47 gene in the test sample is equal to, or lower than, the level of the CD47 gene in the reference sample then treatment of the subject with leukemia with the CTLA4 antagonist will result in clinical benefit in the subject.

In an illustrative embodiment, the sample may include deoxyribonucleic acid (DNA) or ribonucleic acid (RNA). The sample may be from a plasma sample, a blood sample, a marrow sample, a lymph node, or any site of leukemic infection. The sample may also include circulating tumor cells.

In an illustrative embodiment, the reference sample may be obtained from healthy normal tissue, leukemia that received a clinical benefit from CTLA4 antagonist, or leukemia that did not receive a clinical benefit from CTLA4 antagonist.

In an illustrative embodiment, the expression level of the leukemia-associated gene may be detected via an Affymetrix Gene Array hybridization, next generation sequencing, ribonucleic acid sequencing (RNA-seq), a real time reverse transcriptase polymerase chain reaction (real time RT-PCR) assay, immunohistochemistry (IHC), immunofluorescence, or methylation-specific PCR.

In an illustrative embodiment, the expression level of the leukemia-associated gene may be detected via RNA-seq and the reference sample may be obtained from healthy normal tissue from the same individual as the test sample or one or more healthy normal tissues from different individuals.

In an illustrative embodiment, the expression level of the leukemia-associated gene may be detected via RT-PCR and the reference sample may be obtained from the same tissue as the test sample.

In an illustrative embodiment, the subject or patient may be a human.

In an illustrative embodiment, the method may further include a step of treating the subject with a chemotherapeutic agent, radiation therapy, cryotherapy, hormone therapy, or immunotherapy. The chemotherapeutic agent may be dacarbazine, temozolomide, nab-paclitaxel, paclitaxel, cisplatin, or carboplatin, or any combination thereof.

In an illustrative embodiment, the method may further include a step of administering an inhibitor of the CD47 gene, thereby treating the leukemia. In particular, the inhibitor comprises a small molecule inhibitor, RNA interference (RNAi), an antibody, an antibody fragment, an antibody drug conjugate, an aptamer, a chimeric antigen receptor (CAR), a T cell receptor, or any combination thereof In the case of an antibody or antibody fragment, the antibody or fragment may be partially humanized, fully humanized, or chimeric. For example, the antibody or antibody fragment may include a nanobody, an Fab, an Fab′, an (Fab′)2, an Fv, a single-chain variable fragment (ScFv), a diabody, a triabody, a tetrabody, a Bis-scFv, a minibody, an Fab2, an Fab3 fragment, or any combination thereof.

In an illustrative embodiment, the method may also include a step of administering to the subject an anti-CTLA4 antibody, thereby treating the leukemia.

In an illustrative embodiment, the CTLA4 antagonist may be Ipilimumab.

In one aspect, the disclosure provides a composition for predicting no clinical benefit in response to CTLA4 therapy comprising a CD47 molecule (CD47) gene synthesized complementary deoxyribonucleic acid (cDNA).

In an illustrative embodiment, the CD47 gene may be immobilized on a solid support.

In an illustrative embodiment, the CD47 gene may be linked to a detectable label such as, for example, a fluorescent label, a luminescent label, a chemiluminescent label, a radiolabel, a SYBR Green label, or a Cy3-label.

In one aspect, the disclosure provides a method of treating cancer in a subject in need thereof, that may include the step of administering a therapeutically effective amount of one or more CTLA4 inhibitor agents to the subject, where the subject may be identified as not having aberrant expression of at least one resistant cancer-associated gene.

In an illustrative embodiment, the CTLA4 antagonist may be Ipilimumab.

In an illustrative embodiment, the at least one resistant cancer-associated gene may be a CD47 molecule (CD47) gene.

In one aspect, the disclosure provides a method of treating cancer in a subject in need thereof, that may include the following steps: identifying the subject as having aberrant expression of at least one resistant cancer-associated gene; and co-administering a therapeutically effective amount of one or more CTLA4 inhibitor agents and one or more inhibitors of the at least one resistant cancer-associated genes to the subject, thereby treating the cancer.

In an illustrative embodiment, the CTLA4 antagonist may be Ipilimumab.

In an illustrative embodiment, the at least one resistant cancer-associated gene may be a CD47 molecule (CD47) gene.

In an illustrative embodiment, the cancer may be leukemia.

In an illustrative embodiment, the one or more inhibitors may include a small molecule inhibitor, RNA interference (RNAi), an antibody, an antibody fragment, an antibody drug conjugate, an aptamer, a chimeric antigen receptor (CAR), a T cell receptor, or any combination thereof. In the case of an antibody or antibody fragment, the antibody or fragment may be partially humanized, fully humanized, or chimeric. The antibody or antibody fragment may include a nanobody, an Fab, an Fab′, an (Fab′)2, an Fv, a single-chain variable fragment (ScFv), a diabody, a triabody, a tetrabody, a Bis-scFv, a minibody, an Fab2, an Fab3 fragment, or any combination thereof.

In an illustrative embodiment, the method may further include a step of administering to the subject an anti-CTLA4 antibody, thereby treating the leukemia.

In one aspect, the disclosure provides a kit, comprising reagents for assaying a biological sample from a subject with cancer for aberrant expression of at least one resistant cancer-associated gene.

In an illustrative embodiment, the aberrant expression of the at least one resistant cancer-associated gene may be overexpression of the at least one resistant cancer-associated gene.

In an illustrative embodiment, the at least one resistant cancer-associated gene may be a CD47 molecule (CD47) gene.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

The term “antineoplastic agent” is used herein to refer to agents that have the functional property of inhibiting a development or progression of a neoplasm in a human, e.g., a leukemia. Inhibition of metastasis is frequently a property of antineoplastic agents.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ϵ isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (α), delta (δ), epsilon (ϵ), gamma (γ) and mu (μ), respectively. The γ and α classes are further divided into subclasses on the basis of relatively minor differences in CH sequence and function, e.g., humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-101 (H3) in the VH when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al. Nucl. Acids Res. 27: 209-212 (1999), Ruiz, M. e al. Nucl. Acids Res. 28: 219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with AHo; Honneger, A. and Plunkthun, A. J. Mol. Biol. 309: 657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256: 495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc.). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321: 522-525 (1986); Riechmann et al., Nature 332: 323-329 (1988); and Presta, Curr. Op. Struct. Biol. 2: 593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321: 522-525 (1986); Reichmann et al., Nature, 332: 323-327 (1988); Verhoeyen et al., Science, 239: 1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a CL and at least heavy chain constant domains, CH 1, CH 2 and CH 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, FeϵRI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH 1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90: 6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Ka, of greater than or equal to about 104 M-1, or greater than or equal to about 105 M-1, greater than or equal to about 106 M-1, greater than or equal to about 107 M 1, or greater than or equal to 108 M-1. Affinity of an antibody for its cognate antigen is also commonly expressed as a dissociation constant KD, and in certain embodiments, HuM2e antibody specifically binds to M2e if it binds with a KD of less than or equal to 10-4 M, less than or equal to about 10-5 M, less than or equal to about 10-6 M, less than or equal to 10-7 M, or less than or equal to 10-8 M. Affinities of antibodies can be readily determined using conventional techniques, for example, those described by Scatchard et al. (Ann. N.Y. Acad. Sci. USA 51: 660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as 152 Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., leukemia, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152: 399; Kimmel, A. R. (1987) Methods Enzymol. 152: 507).

The terms “isolated,” “purified, ” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By “marker” is meant any protein or polynucleotide having an alteration in expression level or activity that is associated with a disease or disorder, e.g., leukemia.

By “modulate” is meant alter (increase or decrease). Such alterations are detected by standard art-known methods such as those described herein.

The term, “normal amount” refers to a normal amount of a complex in an individual known not to be diagnosed with leukemia. The amount of the molecule can be measured in a test sample and compared to the “normal control level,” utilizing techniques such as reference limits, discrimination limits, or risk defining thresholds to define cutoff points and abnormal values (e.g., for leukemia). The “normal control level” means the level of one or more proteins (or nucleic acids) or combined protein indices (or combined nucleic acid indices) typically found in a subject known not to be suffering from leukemia. Such normal control levels and cutoff points may vary based on whether a molecule is used alone or in a formula combining other proteins into an index. Alternatively, the normal control level can be a database of protein patterns from previously tested subjects who did not convert to leukemia over a clinically relevant time horizon. In another aspect, the normal control level can be a level relative to a housekeeping gene.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196: 180, 1977); Grunstein and Hogness (Proc. Natl. Acad. Sci., USA 72: 3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, in one aspect, “next-generation sequencing” (NGS), also known as high-throughput sequencing, is the catch-all term used to describe a number of different sequencing methodologies including, but not limited to, Illumina® sequencing, Roche 454 sequencing™ Ion torrent™: Proton/personal genome machine (PGM) sequencing, and SOLiD sequencing. These recent technologies allow for sequencing DNA and RNA much more quickly and cheaply than the previously used Sanger sequencing. See, LeBlanc et al., 2015 Cancers, 7: 1925-1958, incorporated herein by reference; and Goodwin et al., 2016 Nature Reviews Genetics, 17: 333-351, incorporated herein by reference.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., leukemia, and of its evolution as well as the prospect of recovery as anticipated from usual course of the disease. Once the aggressiveness (e.g. the Gleason score) has been determined, appropriate methods of treatments are chosen.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from leukemia tumors or the surrounding microenvironment (i.e., the stroma). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The strip may be a nucleic acid-probe (or protein) coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody or nucleic acid molecule). Thus, the support configuration may be spherical, as in a bead, or cylindrical, as in the inside surface of a test tube, or the external surface of a rod. Alternatively, the surface may be flat such as a sheet, or test strip, etc. For example, the supports include polystyrene beads. Those skilled in the art will know many other suitable carriers for binding antibody or antigen, or will be able to ascertain the same by use of routine experimentation. In other aspects, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains a binding agent alone or together with a binding agent for at least one, two, three or more other molecules.

By “specifically binds” is meant a compound or antibody that recognizes and binds a polypeptide of the invention, but which does not substantially recognize and bind other molecules in a sample, for example, a biological sample, which naturally includes a polypeptide of the invention.

A “specific binding agent” describes agents having greater than 10-fold, preferably greater than 100-fold, and most preferably, greater than 1000-fold affinity for the target molecule as compared to another molecule. As the skilled artisan will appreciate the term specific is used to indicate that other biomolecules present in the sample do not significantly bind to the binding agent specific for the target molecule. Preferably, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, an antibody has a binding affinity in the low micromolar (10-6), nanomolar (10-7-10-9), with high affinity antibodies in the low nanomolar (10-9) or pico molar (10-12) range for its specific target molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer (e.g., leukemia) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

As used herein, in one aspect, the “tumor microenvironment” (TME) is the cellular environment in which a tumor exists, including surrounding blood vessels, immune cells, fibroblasts, bone marrow-derived inflammatory cells, lymphocytes, signaling molecules and the extracellular matrix (ECM). The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of cancerous cells, such as in immuno-editing.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the invention.

These and other embodiments are disclosed and/or encompassed by, the following detailed description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1A and FIG. 1B show photographic images of a leukemia cutis associated cutaneous lesion before (FIG. 1A) and after (FIG. 1B) treatment with a single dose of Ipilimumab. FIG. 1C and FIG. 1D show photomicrographs of as histologically stained tissue sections of the cutaneous lesion before (FIG. 1C) and after (FIG. 1D) treatment;

FIG. 2 depicts a schematic of the Cancer-Immunity Cycle, which is a theoretical construct that describes the iterative steps that occur during the generation of an effective anti-cancer immune response;

FIG. 3 shows a bar graph depicting an increase in expression of genes responsible for immune cell trafficking after Ipilimumab treatment in both responding and nonresponding tumors as indicated by the difference in expression levels pre- and post-treatment. The left and right axes both show units of gene expression: transcripts per million (TPM)—the standard used to measure gene expression units from RNA sequencing data (Li B, et al. Bioinformatics 26: 4 2010). The right axis is scaled for CXCL9 and CXCL10 gene expression; whereas the left axis describes CCL3, CCL4, CCR5, and CX3L1. The left two pre/post labels correspond to a single patient (with biological replicate samples/biopsies; in black) with the green labels correspond to a second patient with complete response. Expression levels were tested for the following genes: C-X-C Motif Chemokine Ligand 9 (CXCL9; shown in black), C-X-C Motif Chemokine Ligand 10 (CXCL10; shown in orange), C-C Motif Chemokine Ligand 3 (CCL3; shown in maroon), C-C Motif Chemokine Ligand 4 (CCL4; shown in purple, C-C Motif Chemokine Receptor 5 (CCR5; shown in blue), and C-X3-C Motif Chemokine Ligand 1 (CX3CL1; shown in green). As can be seen in the bar graph, pre-Ipilimumab tumor in the relapsing patient has a very high ‘inflamed’ baseline of chemokines that is downregulated in the post-Ipilimumab relapsed tumor.

FIG. 4 depicts a bar graph showing an increase in genes responsible for immune cell trafficking through the vascular endothelium after Ipilimumab treatment in both responding and nonresponding tumors. The left and right axes both show units of gene expression: transcripts per million (TPM)—the standard used to measure gene expression units from RNA sequencing data (Li B, et al. Bioinformatics 26: 4 2010). The right axis is scaled for ICAM1 and VCAM1 gene expression. The left two pre/post labels correspond to a single patient (with biological replicate samples/biopsies; in black) with the green labels correspond to a second patient with complete response. Expression levels were tested for the following genes: Intercellular Adhesion Molecule 1 (ICAM1; shown in black) and Vascular Cell Adhesion Molecule 1 (VCAM1; shown in gray). As can be seen in the bar graph, pre-Ipilimumab tumor in the relapsing patient has a very high ‘inflamed’ baseline of chemokines that is downregulated in the post-Ipilimumab relapsed tumor.

FIG. 5 depicts six bar graphs that show gene expression patterns in specific immune sub-populations corresponding to T cells, B cells, and Macrophages, respectively. After treatment with Ipilimumab, CD8A expression is increased in T cells, CD20 and CD138 expression is increased in B cells and plasma cells, respectively, and the expression of MRC1 and CD163 and Chemerin is increased in macrophages. However, in the non-responding patient increased CD8+ expression (blue bars) is only seen in T Cells after treatment with Ipilimumab. In the case of a relapse, all of the cell types (i.e., T cells, B cells, and Macrophages) are down-regulated (red bars).

FIG. 6 depicts a graph showing macrophage evasion in responder, relapse, and non-responder patients, respectively. CD47 expression was analyzed to assess macrophage defense capabilities. As shown, the three resistant tumors (“Rel,” “Pre,” and “Post from the non-responder) are the ones with the highest levels of CD47 expression, and they are also the ones with the lowest macrophages gene expression.

FIG. 7 depicts two graphs that show data assessing leukemic recognition by T cells. As shown in the left graph, responders are associated with T cells that express T cell receptor genes such as CD3E, CD3D, CD3G, and CD247, while these genes are not expressed in the relapse or non-responders. Similarly, responders are associated with T cells that express signaling genes such as LCK, ITK, and ZAP70, while these genes are not expressed in the relapse or non-responders (right graph). The relapse category is associated with downregulation of both the T cell receptor genes and the signaling genes.

FIG. 8 depicts two graphs that show data assessing T cell activation. As shown in the left graph, responders are associated with T cells that express coinhibitory receptor genes such as CTLA4, LAG3, TIGIT, HAVCR2, and PD1, while these genes are not expressed in the relapse or non-responders. Similarly, responders are associated with T cells that express costimulatory receptor genes such as ICOS, CD28, and CD27, while these genes are not expressed in the relapse or non-responders (right graph).

FIG. 9 depicts a graph that show data assessing whether T cells are activated and cytolytic. Responders are associated with T cells that express CD8A and perforin (PRF1), indicating that their tumors are being infiltrated by CD8 T cells that are cytolytic and have the capacity to kill. Although CD8 T cells come to the tumor in the nonresponding patient after Ipilimumab treatment, these T cells are not cytolytic, as evidenced by lack of PRF1 gene expression.

FIG. 10 depicts immunohistochemistry staining seven days before (left) and twelve days after (right) treatment with Ipilimumab in a responder patient. The staining data shows that CD8 T cells come into the tumor after Ipilimumab treatment (e.g., PRF1 staining) and contacts and kills multiple tumor cells.

### DETAILED DESCRIPTION OF THE INVENTION

The invention is based, at least in part, upon the identification of gene expression patterns that discriminate the clinical outcomes of CTLA4 antagonists in neoplasia (e.g., leukemia). In particular, the techniques herein provide gene expression patterns/signatures that identify forms of leukemia that may be resistant to treatment with CTLA4 antagonists such as, for example, Ipilimumab. Prior to the invention described herein, the skilled artisan was not aware of any molecular signatures capable of precisely predicting response and resistance to CTLA4 antagonists.

Ipilimumab is an FDA-approved antibody targeting the CTLA4 pathway. Ipilimumab shows an overall survival benefit in leukemia and can induce durable tumor remissions in some patients. However, many leukemia patients do not respond to Ipilimumab treatment because they are resistant to the CTLA4 antagonist. Prior to the invention described herein, there was no way to predict clinical outcome. Because Ipilimumab carries significant autoimmune toxicity, predicting who will and will not benefit is of critical clinical importance. Ipilimumab is falling out of clinical use with the approval of newer, less toxic immunotherapies; however, long term survival data is only available for this agent. Thus, the results presented herein allow for precisely pairing CTLA4 blockade therapy with the appropriate patient, and determining whether certain patients may benefit from combination therapy (e.g., Ipilimumab and CD47 antibody).

### Leukemia

Cancer starts when cells in the body begin to grow out of control. Cells in nearly any part of the body can become cancerous, and may then spread to other areas of the body (e.g., metastasize). Leukemia refers to a group of cancers that usually begin in the bone marrow. Leukemia usually results in high numbers of abnormal white blood cells that are not fully developed (e.g., blasts or leukemia cells). Symptoms of leukemia may include bleeding and bruising, fatigue, fever, and an increased risk of infections, and are usually due to a lack of normal blood cells. Diagnosis is typically made by blood tests or bone marrow biopsy. Different types of leukemia may have different causes, and both genetic and environmental risk factors may be involved.

There are four main types of leukemia: 1) acute lymphoblastic leukemia (ALL), 2) acute myeloid leukemia (AML), 3) chronic lymphocytic leukemia (CLL), and 4) chronic myeloid leukemia (CML). Leukemias and lymphomas both belong to a broader group of tumors that affect the blood, bone marrow, and lymphoid system, known as tumors of the hematopoietic and lymphoid tissues. Specific types of leukemia may include, but are not limited to, the following:

Acute lymphoblastic leukemia (ALL) is the most common type of leukemia in young children, but may also affects adults, especially those 65 and older. Standard treatments involve chemotherapy and radiotherapy, and the survival rates vary by age: 85% in children and 50% in adults. Subtypes include precursor B acute lymphoblastic leukemia, precursor T acute lymphoblastic leukemia, Burkitt's leukemia, and acute biphenotypic leukemia.

Chronic lymphocytic leukemia (CLL) most often affects adults over the age of 55. It rarely affects children. Two-thirds of affected people are men. The five-year survival rate is 75%. It is incurable, but there are many effective treatments. B-cell prolymphocytic leukemia is a sub-type of CLL that is a more aggressive disease.

Acute myelogenous leukemia (AML) occurs more commonly in adults than in children, and more commonly in men than women. It is generally treated with chemotherapy. The five-year survival rate is 40%, except for Acute Promyelocytic Leukemia (APL), which has a survival rate greater than 90%. Subtypes of AML include acute promyelocytic leukemia, acute myeloblastic leukemia, and acute megakaryoblastic leukemia.

Chronic myelogenous leukemia (CML) occurs primarily in adults, although a very small number of children may also develop CML. CML is usually treated with imatinib (Gleevec in United States, Glivec in Europe) or other drugs, and the five-year survival rate is 90%. Chronic myelomonocytic leukemia is a sub-type of CML.

Hairy cell leukemia (HCL) is sometimes considered a subset of chronic lymphocytic leukemia. About 80% of affected people are adult men, and to date no cases in children have been reported. HCL is incurable but easily treatable. Survival is 96% to 100% at ten years.

T-cell prolymphocytic leukemia (T-PLL) is a very rare and aggressive form of adult leukemia. Despite its overall rarity, it is the most common type of mature T cell leukemia, as nearly all other leukemias involve B cells. It is difficult to treat, and the median survival is short (e.g., months).

Large granular lymphocytic leukemia may involve either T-cells or NK cells. This form of leukemia is rare and not particularly aggressive.

Adult T-cell leukemia is caused by human T-lymphotropic virus (HTLV), a virus similar to HIV. HTLV infects CD4+ T-cells and replicates within them; however, it does not destroy the T-cells. Instead, HTLV “immortalizes” the infected T-cells, giving them the ability to proliferate abnormally. Human T-cell lymphotropic virus types I and II (HTLV-I/II) are endemic in certain areas of the world.

Most forms of leukemia are treated with pharmaceutical medication, typically combined into a multi-drug chemotherapy regimen. Some are also treated with radiation therapy. In some cases, a bone marrow transplant is effective.

### CTLA-4-Blockade

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells (Tregs), but only upregulated in conventional T cells after activation. CTLA4 acts as an “off” switch when bound to CD80 or CD86 on the surface of antigen-presenting cells. Recent reports suggest that blocking CTLA4 (using antagonistic antibodies against CTLA such as Ipilimumab) results in therapeutic benefit. CTLA4 blockade inhibits immune system tolerance to tumors and provides a useful immunotherapy strategy for patients with cancer. See, e.g., Grosso J. and Jure-Kunkel M. 2013, Cancer Immun., 13: 5, incorporated herein by reference.

Ipilimumab, a fully human monoclonal antibody specific to CTLA-4, improves overall survival in metastatic melanoma patients (Ji et al., 2012 Cancer Immunol Immunother, 61: 1019-1031, incorporated herein by reference). Indeed, monoclonal antibodies directed against CTLA4, such as Ipilimumab, yield considerable clinical benefit for patients with metastatic melanoma by inhibiting checkpoint activity; however, prior to the invention described herein, clinical predictors of response to these therapies were incompletely characterized (Van Allen, et al., 2015 Science, 350(6257): 207-211, incorporated herein by reference). See also, Snyder et al., 2014 The New England Journal of Medicine, 373(20): 1984, incorporated herein by reference.

### World Health Organization (WHO) Criteria

The WHO Criteria for evaluating the effectiveness of anti-cancer agents on tumor shrinkage, developed in the 1970s by the International Union Against Cancer and the World Health Organization, represented the first generally agreed specific criteria for the codification of tumor response evaluation. These criteria were first published in 1981 (Miller et al., 1981 Clin Cancer Res., 47(1): 207-14, incorporated herein by reference). WHO Criteria proposed >50% tumor shrinkage for a Partial Response and >25% tumor increase for Progressive Disease.

### Response Evaluation Criteria in Solid Tumors (RECIST)

RECIST is a set of published rules that define when tumors in cancer patients improve (“respond”), stay the same (“stabilize”), or worsen (“progress”) during treatment (Eisenhauer et al., 2009 European Journal of Cancer, 45: 228-247, incorporated herein by reference). Only patients with measureable disease at baseline should be included in protocols where objective tumor response is the primary endpoint.

The response criteria for evaluation of target lesions are as follows:
Complete Response (CR): Disappearance of all target lesions.
Partial Response (PR): At least a 30% decrease in the sum of the longest diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum of the LD of target lesions, taking as reference the smallest sum LD recorded since the treatment started or the appearance of one or more new lesions.

The response criteria for evaluation of non-target lesions are as follows:
Complete Response (CR): Disappearance of all non-target lesions and normalization of tumor marker level.
Incomplete Response/Stable Disease (SD): Persistence of one or more non-target lesion(s) or/and maintenance of tumor marker level above the normal limits.
Progressive Disease (PD): Appearance of one or more new lesions and/or unequivocal progression of existing non-target lesions.

The response criteria for evaluation of best overall response are as follows. The best overall response is the best response recorded from the start of the treatment until disease progression/recurrence (taking as reference for PD the smallest measurements recorded since the treatment started). In general, the patient's best response assignment will depend on the achievement of both measurement and confirmation criteria.
Patients with a global deterioration of health status requiring discontinuation of treatment without objective evidence of disease progression at that time should be classified as having “symptomatic deterioration”. Every effort should be made to document the objective progression even after discontinuation of treatment.
In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Immune-Related Response Criteria

The immune-related response criteria (irRC) is a set of published rules that define when tumors in cancer patients improve (“respond”), stay the same (“stabilize”), or worsen (“progress”) during treatment, where the compound being evaluated is an immuno-oncology drug. The Immune-Related Response Criteria, first published in 2009 (Wolchok et al., 2009 Clin Cancer Res, 15(23): 7412, incorporated herein by reference), arose out of observations that immuno-oncology drugs would fail in clinical trials that measured responses using the WHO or RECIST Criteria, because these criteria could not account for the time gap in many patients between initial treatment and the apparent action of the immune system to reduce the tumor burden. The key driver in the development of the irRC was the observation that, in studies of various cancer therapies derived from the immune system such as cytokines and monoclonal antibodies, the looked-for Complete and Partial Responses as well as Stable Disease only occurred after an increase in tumor burden that the conventional RECIST Criteria would have dubbed ‘Progressive Disease.’ RECIST failed to take account of the delay between dosing and an observed anti-tumor T cell response, so that otherwise ‘successful’ drugs—that is, drugs which ultimately prolonged life—failed in clinical trials.

The irRC are based on the WHO Criteria; however, the measurement of tumor burden and the assessment of immune-related response have been modified as set forth below.

### Measurement of Tumor Burden

In the irRC, tumor burden is measured by combining ‘index’ lesions with new lesions. Ordinarily, tumor burden would be measured with a limited number of ‘index’ lesions (that is, the largest identifiable lesions) at baseline, with new lesions identified at subsequent time points counting as ‘Progressive Disease’. In the irRC, by contrast, new lesions are a change in tumor burden. The irRC retained the bidirectional measurement of lesions that had originally been laid down in the WHO Criteria.

### Assessment of Immune-Related Response

In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). Even if tumor burden is rising, the immune system is likely to “kick in” some months after first dosing and lead to an eventual decline in tumor burden for many patients. The 25% threshold accounts for this apparent delay.

### Gene Expression Profiling

In general, methods of gene expression profiling may be divided into two large groups: methods based on hybridization analysis of polynucleotides and methods based on sequencing of polynucleotides. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse-transcribed using a GeneAmp RNA PCR kit (Perkin Elmer, California, USA), following the manufacturer's instructions. The cDNA is then used as template in a subsequent PCR amplification and quantitative analysis using, for example, a TaqMan RTM (Life Technologies, Inc., Grand Island, N.Y.) assay.

### Microarrays

Differential gene expression can also be identified, or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., leukemia tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al., Proc. Natl. Acad. Sci. USA 93(2): 106-149 (1996)). In various configurations, microarray analysis is performed by commercially available equipment, following manufacturer's protocols, such as by using the Affymetrix GenChip technology, or Incyte's microarray technology.

### RNA-Seq

RNA sequencing (RNA-seq), also called whole transcriptome shotgun sequencing (WTSS), uses next-generation sequencing (NGS) to reveal the presence and quantity of RNA in a biological sample at a given moment in time.

RNA-Seq is used to analyze the continually changing cellular transcriptome. See, e.g., Wang et al., 2009 Nat Rev Genet, 10(1): 57-63, incorporated herein by reference. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and needing to know the sequence of interest. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, NGS of cDNA (notably RNA-Seq).

By “C-X-C Motif Chemokine Ligand 9 (CXCL9) nucleic acid molecule” is meant a polynucleotide encoding a CXCL9 polypeptide. An exemplary CXCL9 nucleic acid molecule is provided at NCBI Accession No. NM_002416.2, incorporated herein by reference, and reproduced below (SEQ ID NO: 1):

| | |
|---|---|
| 1 | aaaatgtgtt ctctaaagaa tttctcaggc tcaaaatcca atacaggagt gacttggaac |
| 61 | tccattctat cactatgaag aaaagtggtg ttcttttcct cttgggcatc atcttgctgg |
| 121 | ttctgattgg agtgcaagga accccagtag tgagaaaggg tcgctgttcc tgcatcagca |
| 181 | ccaaccaagg gactatccac ctacaatcct tgaaagacct taaacaattt gccccaagcc |
| 241 | cttcctgcga gaaaattgaa atcattgcta cactgaagaa tggagttcaa acatgtctaa |
| 301 | acccagattc agcagatgtg aaggaactga ttaaaaagtg ggagaaacag gtcagccaaa |
| 361 | agaaaaagca aaagaatggg aaaaaacatc aaaaaaagaa agttctgaaa gttcgaaaat |
| 421 | ctcaacgttc tcgtcaaaag aagactacat aagagaccac ttcaccaata agtattctgt |
| 481 | gttaaaaatg ttctatttta attataccgc tatcattcca aaggaggatg gcatataata |
| 541 | caaaggctta ttaatttgac tagaaaattt aaaacattac tctgaaattg taactaaagt |
| 601 | tagaaagttg attttaagaa tccaaacgtt aagaattgtt aaaggctatg attgtctttg |
| 661 | ttcttctacc acccaccagt tgaatttcat catgcttaag gccatgattt tagcaatacc |
| 721 | catgtctaca cagatgttca cccaaccaca tcccactcac aacagctgcc tggaagagca |
| 781 | gccctaggct tccacgtact gcagcctcca gagagtatct gaggcacatg tcagcaagtc |
| 841 | ctaagcctgt tagcatgctg gtgagccaag cagtttgaaa ttgagctgga cctcaccaag |
| 901 | ctgctgtggc catcaacctc tgtatttgaa tcagcctaca ggcctcacac acaatgtgtc |
| 961 | tgagagattc atgctgattg ttattgggta tcaccactgg agatcaccag tgtgtggctt |
| 1021 | tcagagcctc ctttctggct ttggaagcca tgtgattcca tcttgcccgc tcaggctgac |
| 1081 | cactttattt ctttttgttc ccctttgctt cattcaagtc agctcttctc catcctacca |
| 1141 | caatgcagtg cctttcttct ctccagtgca cctgtcatat gctctgattt atctgagtca |
| 1201 | actcctttct catcttgtcc ccaacacccc acagaagtgc tttcttctcc caattcatcc |
| 1261 | tcactcagtc cagcttagtt caagtcctgc ctcttaaata aacctttttg gacacacaaa |
| 1321 | ttatcttaaa actcctgttt cacttggttc agtaccacat gggtgaacac tcaatggtta |
| 1381 | actaattctt gggtgtttat cctatctctc caaccagatt gtcagctcct tgagggcaag |
| 1441 | agccacagta tatttccctg tttcttccac agtgcctaat aatactgtgg aactaggttt |
| 1501 | taataatttt ttaattgatg ttgttatggg caggatggca accagaccat tgtctcagag |
| 1561 | caggtgctgg ctctttcctg gctactccat gttggctagc ctctggtaac ctcttactta |
| 1621 | ttatcttcag gacactcact acagggacca gggatgatgc aacatccttg tctttttatg |
| 1681 | acaggatgtt tgctcagctt ctccaacaat aagaagcacg tggtaaaaca cttgcggata |
| 1741 | ttctggactg tttttaaaaa atatacagtt taccgaaaat catataatct tacaatgaaa |
| 1801 | aggactttat agatcagcca gtgaccaacc ttttcccaac catacaaaaa ttccttttcc |
| 1861 | cgaaggaaaa gggctttctc aataagcctc agctttctaa gatctaacaa gatagccacc |
| 1921 | gagatcctta tcgaaactca ttttaggcaa atatgagttt tattgtccgt ttacttgttt |
| 1981 | cagagtttgt attgtgatta tcaattacca caccatctcc catgaagaaa gggaacggtg |
| 2041 | aagtactaag cgctagagga agcagccaag tcggttagtg gaagcatgat tggtgcccag |
| 2101 | ttagcctctg caggatgtgg aaacctcctt ccaggggagg ttcagtgaat tgtgtaggag |
| 2161 | aggttgtctg tggccagaat ttaaacctat actcactttc ccaaattgaa tcactgctca |
| 2221 | cactgctgat gatttagagt gctgtccggt ggagatccca cccgaacgtc ttatctaatc |
| 2281 | atgaaactcc ctagttcctt catgtaactt ccctgaaaaa tctaagtgtt tcataaattt |
| 2341 | gagagtctgt gacccactta ccttgcatct cacaggtaga cagtatataa ctaacaacca |
| 2401 | aagactacat attgtcactg acacacacgt tataatcatt tatcatatat atacatacat |
| 2461 | gcatacactc tcaaagcaaa taatttttca cttcaaaaca gtattgactt gtataccttg |
| 2521 | taatttgaaa tattttcttt gttaaaatag aatggtatca ataaatagac cattaatcag |
| 2581 | aaaacagatc ttgatttttt ttctcttgaa tgtacccttc aactgttgaa tgtttaatag |
| 2641 | taaatcttat atgtccttat ttacttttta gctttctctc aaataaagtg taacactagt |
| 2701 | tgagataaaa aaaaaaaaaa aaa |

By “CXCL9 polypeptide” is meant a polypeptide or fragment thereof having at least about 85% amino acid identity to NCBI Accession No. NP_002407.1, incorporated herein by reference, and having chemokine activity, as reproduced below (SEQ ID NO: 2):

| | |
|---|---|
| 1 | mkksgvlfll giillvligv qgtpvvrkgr cscistnqgt ihlqslkdlk qfapspscek |
| 61 | ieiiatlkng vqtclnpdsa dvkelikkwe kqvsqkkkqk ngkkhqkkkv lkvrksqrsr |
| 121 | qkktt |

By “C-X-C Motif Chemokine Ligand 10 (CXCL10) nucleic acid molecule” is meant a polynucleotide encoding a CXCL10 polypeptide. An exemplary CXCL10 nucleic acid molecule is provided at NCBI Accession No. NM_001565.3, incorporated herein by reference, and reproduced below (SEQ ID NO: 3):

| | |
|---|---|
| 1 | ctttgcagat aaatatggca cactagcccc acgttttctg agacattcct caattgctta |
| 61 | gacatattct gagcctacag cagaggaacc tccagtctca gcaccatgaa tcaaactgcc |
| 121 | attctgattt gctgccttat ctttctgact ctaagtggca ttcaaggagt acctctctct |
| 181 | agaactgtac gctgtacctg catcagcatt agtaatcaac ctgttaatcc aaggtcttta |
| 241 | gaaaaacttg aaattattcc tgcaagccaa ttttgtccac gtgttgagat cattgctaca |
| 301 | atgaaaaaga agggtgagaa gagatgtctg aatccagaat cgaaggccat caagaattta |
| 361 | ctgaaagcag ttagcaagga aaggtctaaa agatctcctt aaaaccagag gggagcaaaa |
| 421 | tcgatgcagt gcttccaagg atggaccaca cagaggctgc ctctcccatc acttccctac |
| 481 | atggagtata tgtcaagcca taattgttct tagtttgcag ttacactaaa aggtgaccaa |
| 541 | tgatggtcac caaatcagct gctactactc ctgtaggaag gttaatgttc atcatcctaa |
| 601 | gctattcagt aataactcta ccctggcact ataatgtaag ctctactgag gtgctatgtt |
| 661 | cttagtggat gttctgaccc tgcttcaaat atttccctca cctttcccat cttccaaggg |
| 721 | tactaaggaa tctttctgct ttggggttta tcagaattct cagaatctca aataactaaa |
| 781 | aggtatgcaa tcaaatctgc tttttaaaga atgctcttta cttcatggac ttccactgcc |
| 841 | atcctcccaa ggggcccaaa ttctttcagt ggctacctac atacaattcc aaacacatac |
| 901 | aggaaggtag aaatatctga aaatgtatgt gtaagtattc ttatttaatg aaagactgta |
| 961 | caaagtagaa gtcttagatg tatatatttc ctatattgtt ttcagtgtac atggaataac |
| 1021 | atgtaattaa gtactatgta tcaatgagta acaggaaaat tttaaaaata cagatagata |
| 1081 | tatgctctgc atgttacata agataaatgt gctgaatggt tttcaaaata aaaatgaggt |
| 1141 | actctcctgg aaatattaag aaagactatc taaatgttga aagatcaaaa ggttaataaa |
| 1201 | gtaattataa ctaagaaaaa aaaaaaa |

By “CXCL10 polypeptide” is meant a polypeptide or fragment thereof having at least about 85% amino acid identity to NCBI Accession No. NP_001556.2, incorporated herein by reference, and having chemokine activity, as reproduced below (SEQ ID NO: 4):

| | |
|---|---|
| 1 | mnqtailicc lifltlsgiq gvplsrtvrc tcisisnqpv nprsleklei ipasqfcpry |
| 61 | eiiatmkkkg ekrclnpesk aiknllkavs kerskrsp |

By “C-C Motif Chemokine Ligand 3 (CCL3) nucleic acid molecule” is meant a polynucleotide encoding a CCL3 polypeptide. An exemplary CCL3 nucleic acid molecule is provided at NCBI Accession No. NM_002983.2, incorporated herein by reference, and reproduced below (SEQ ID NO: 5):

| | |
|---|---|
| 1 | agctggtttc agacttcaga aggacacggg cagcagacag tggtcagtcc tttcttggct |
| 61 | ctgctgacac tcgagcccac attccgtcac ctgctcagaa tcatgcaggt ctccactgct |
| 121 | gcccttgctg tcctcctctg caccatggct ctctgcaacc agttctctgc atcacttgct |
| 181 | gctgacacgc cgaccgcctg ctgcttcagc tacacctccc ggcagattcc acagaatttc |
| 241 | atagctgact actttgagac gagcagccag tgctccaagc ccggtgtcat cttcctaacc |
| 301 | aagcgaagcc ggcaggtctg tgctgacccc agtgaggagt gggtccagaa atatgtcagc |
| 361 | gacctggagc tgagtgcctg aggggtccag aagcttcgag gcccagcgac ctcggtgggc |
| 421 | ccagtgggga ggagcaggag cctgagcctt gggaacatgc gtgtgacctc cacagctacc |
| 481 | tcttctatgg actggttgtt gccaaacagc cacactgtgg gactcttctt aacttaaatt |
| 541 | ttaatttatt tatactattt agtttttgta atttattttc gatttcacag tgtgtttgtg |
| 601 | attgtttgct ctgagagttc ccctgtcccc tcccccttcc ctcacaccgc gtctggtgac |
| 661 | aaccgagtgg ctgtcatcag cctgtgtagg cagtcatggc accaaagcca ccagactgac |
| 721 | aaatgtgtat cggatgcttt tgttcagggc tgtgatcggc ctggggaaat aataaagatg |
| 781 | ctcttttaaa aggtaaaaaa aaaaaaaaaa aaa |

By “CCL3 polypeptide” is meant a polypeptide or fragment thereof having at least about 85% amino acid identity to NCBI Accession No. NP_002974.1, incorporated herein by reference, and having chemokine activity, as reproduced below (SEQ ID NO: 6):

| | |
|---|---|
| 1 | mqvstaalav llctmalcnq fsaslaadtp taccfsytsr qipqnfiady fetssqcskp |
| 61 | gvifltkrsr qvcadpseew vqkyvsdlel sa |

By “C-C Motif Chemokine Ligand 4 (CCL4) nucleic acid molecule” is meant a polynucleotide encoding a CCL4 polypeptide. An exemplary CCL4 nucleic acid molecule is provided at NCBI Accession No. NM_002984.3, incorporated herein by reference, and reproduced below (SEQ ID NO: 7):

| | |
|---|---|
| 1 | agcacaggac acagctgggt tctgaagctt ctgagttctg cagcctcacc tctgagaaaa |
| 61 | cctcttttcc accaatacca tgaagctctg cgtgactgtc ctgtctctcc tcatgctagt |
| 121 | agctgccttc tgctctccag cgctctcagc accaatgggc tcagaccctc ccaccgcctg |
| 181 | ctgcttttct tacaccgcga ggaagcttcc tcgcaacttt gtggtagatt actatgagac |
| 241 | cagcagcctc tgctcccagc cagctgtggt attccaaacc aaaagaagca agcaagtctg |
| 301 | tgctgatccc agtgaatcct gggtccagga gtacgtgtat gacctggaac tgaactgagc |
| 361 | tgctcagaga caggaagtct tcagggaagg tcacctgagc ccggatgctt ctccatgaga |
| 421 | cacatctcct ccatactcag gactcctctc cgcagttcct gtcccttctc ttaatttaat |
| 481 | cttttttatg tgccgtgtta ttgtattagg tgtcatttcc attatttata ttagtttagc |
| 541 | caaaggataa gtgtccccta tggggatggt ccactgtcac tgtttctctg ctgttgcaaa |
| 601 | tacatggata acacatttga ttctgtgtgt tttcataata aaactttaaa ataaaatgca |
| 661 | gacagtt |

By “CCL4 polypeptide” is meant a polypeptide or fragment thereof having at least about 85% amino acid identity to NCBI Accession No. NP_002975.1, incorporated herein by reference, and having chemokine activity, as reproduced below (SEQ ID NO: 8):

| | |
|---|---|
| 1 | mklcvtvlsl lmlvaafcsp alsapmgsdp ptaccfsyta rklprnfvvd yyetsslcsq |
| 61 | pavvfqtkrs kqvcadpses wvqeyvydle ln |

By “C-C Motif Chemokine Receptor 5 (CCR5) nucleic acid molecule” is meant a polynucleotide encoding a CCR5 polypeptide. An exemplary CCR5 nucleic acid molecule is provided at NCBI Accession No. NM_000579.3, incorporated herein by reference, and reproduced below (SEQ ID NO: 9):

| | |
|---|---|
| 1 | cttcagatag attatatctg gagtgaagaa tcctgccacc tatgtatctg gcatagtatt |
| 61 | ctgtgtagtg ggatgagcag agaacaaaaa caaaataatc cagtgagaaa agcccgtaaa |
| 121 | taaaccttca gaccagagat ctattctcta gcttatttta agctcaactt aaaaagaaga |
| 181 | actgttctct gattcttttc gccttcaata cacttaatga tttaactcca ccctccttca |
| 241 | aaagaaacag catttcctac ttttatactg tctatatgat tgatttgcac agctcatctg |
| 301 | gccagaagag ctgagacatc cgttccccta caagaaactc tccccgggtg gaacaagatg |
| 361 | gattatcaag tgtcaagtcc aatctatgac atcaattatt atacatcgga gccctgccaa |
| 421 | aaaatcaatg tgaagcaaat cgcagcccgc ctcctgcctc cgctctactc actggtgttc |
| 481 | atctttggtt ttgtgggcaa catgctggtc atcctcatcc tgataaactg caaaaggctg |
| 541 | aagagcatga ctgacatcta cctgctcaac ctggccatct ctgacctgtt tttccttctt |
| 601 | actgtcccct tctgggctca ctatgctgcc gcccagtggg actttggaaa tacaatgtgt |
| 661 | caactcttga cagggctcta ttttataggc ttcttctctg gaatcttctt catcatcctc |
| 721 | ctgacaatcg ataggtacct ggctgtcgtc catgctgtgt ttgctttaaa agccaggacg |
| 781 | gtcacctttg gggtggtgac aagtgtgatc acttgggtgg tggctgtgtt tgcgtctctc |
| 841 | ccaggaatca tctttaccag atctcaaaaa gaaggtcttc attacacctg cagctctcat |
| 901 | tttccataca gtcagtatca attctggaag aatttccaga cattaaagat agtcatcttg |
| 961 | gggctggtcc tgccgctgct tgtcatggtc atctgctact cgggaatcct aaaaactctg |
| 1021 | cttcggtgtc gaaatgagaa gaagaggcac agggctgtga ggcttatctt caccatcatg |
| 1081 | attgtttatt ttctcttctg ggctccctac aacattgtcc ttctcctgaa caccttccag |
| 1141 | gaattctttg gcctgaataa ttgcagtagc tctaacaggt tggaccaagc tatgcaggtg |
| 1201 | acagagactc ttgggatgac gcactgctgc atcaacccca tcatctatgc ctttgtcggg |
| 1261 | gagaagttca gaaactacct cttagtcttc ttccaaaagc acattgccaa acgcttctgc |
| 1321 | aaatgctgtt ctattttcca gcaagaggct cccgagcgag caagctcagt ttacacccga |
| 1381 | tccactgggg agcaggaaat atctgtgggc ttgtgacacg gactcaagtg ggctggtgac |
| 1441 | ccagtcagag ttgtgcacat ggcttagttt tcatacacag cctgggctgg gggtggggtg |
| 1501 | ggagaggtct tttttaaaag gaagttactg ttatagaggg tctaagattc atccatttat |
| 1561 | ttggcatctg tttaaagtag attagatctt ttaagcccat caattataga aagccaaatc |
| 1621 | aaaatatgtt gatgaaaaat agcaaccttt ttatctcccc ttcacatgca tcaagttatt |
| 1681 | gacaaactct cccttcactc cgaaagttcc ttatgtatat ttaaaagaaa gcctcagaga |
| 1741 | attgctgatt cttgagttta gtgatctgaa cagaaatacc aaaattattt cagaaatgta |
| 1801 | caacttttta cctagtacaa ggcaacatat aggttgtaaa tgtgtttaaa acaggtcttt |
| 1861 | gtcttgctat ggggagaaaa gacatgaata tgattagtaa agaaatgaca cttttcatgt |
| 1921 | gtgatttccc ctccaaggta tggttaataa gtttcactga cttagaacca ggcgagagac |
| 1981 | ttgtggcctg ggagagctgg ggaagcttct taaatgagaa ggaatttgag ttggatcatc |
| 2041 | tattgctggc aaagacagaa gcctcactgc aagcactgca tgggcaagct tggctgtaga |
| 2101 | aggagacaga gctggttggg aagacatggg gaggaaggac aaggctagat catgaagaac |
| 2161 | cttgacggca ttgctccgtc taagtcatga gctgagcagg gagatcctgg ttggtgttgc |
| 2221 | agaaggttta ctctgtggcc aaaggagggt caggaaggat gagcatttag ggcaaggaga |
| 2281 | ccaccaacag ccctcaggtc agggtgagga tggcctctgc taagctcaag gcgtgaggat |
| 2341 | gggaaggagg gaggtattcg taaggatggg aaggagggag gtattcgtgc agcatatgag |
| 2401 | gatgcagagt cagcagaact ggggtggatt tgggttggaa gtgagggtca gagaggagtc |
| 2461 | agagagaatc cctagtcttc aagcagattg gagaaaccct tgaaaagaca tcaagcacag |
| 2521 | aaggaggagg aggaggttta ggtcaagaag aagatggatt ggtgtaaaag gatgggtctg |
| 2581 | gtttgcagag cttgaacaca gtctcaccca gactccaggc tgtctttcac tgaatgcttc |
| 2641 | tgacttcata gatttccttc ccatcccagc tgaaatactg aggggtctcc aggaggagac |
| 2701 | tagatttatg aatacacgag gtatgaggtc taggaacata cttcagctca cacatgagat |
| 2761 | ctaggtgagg attgattacc tagtagtcat ttcatgggtt gttgggagga ttctatgagg |
| 2821 | caaccacagg cagcatttag cacatactac acattcaata agcatcaaac tcttagttac |
| 2881 | tcattcaggg atagcactga gcaaagcatt gagcaaaggg gtcccataga ggtgagggaa |
| 2941 | gcctgaaaaa ctaagatgct gcctgcccag tgcacacaag tgtaggtatc attttctgca |
| 3001 | tttaaccgtc aataggcaaa ggggggaagg gacatattca tttggaaata agctgccttg |
| 3061 | agccttaaaa cccacaaaag tacaatttac cagcctccgt atttcagact gaatgggggt |
| 3121 | ggggggggcg ccttaggtac ttattccaga tgccttctcc agacaaacca gaagcaacag |
| 3181 | aaaaaatcgt ctctccctcc ctttgaaatg aatatacccc ttagtgtttg ggtatattca |
| 3241 | tttcaaaggg agagagagag gtttttttct gttctgtctc atatgattgt gcacatactt |
| 3301 | gagactgttt tgaatttggg ggatggctaa aaccatcata gtacaggtaa ggtgagggaa |
| 3361 | tagtaagtgg tgagaactac tcagggaatg aaggtgtcag aataataaga ggtgctactg |
| 3421 | actttctcag cctctgaata tgaacggtga gcattgtggc tgtcagcagg aagcaacgaa |
| 3481 | gggaaatgtc tttccttttg ctcttaagtt gtggagagtg caacagtagc ataggaccct |
| 3541 | accctctggg ccaagtcaaa gacattctga catcttagta tttgcatatt cttatgtatg |
| 3601 | tgaaagttac aaattgcttg aaagaaaata tgcatctaat aaaaaacacc ttctaaaata |
| 3661 | aaaaaaaaaa aaaaaaaaaa aaaaaa |

By “CCR5 polypeptide” is meant a polypeptide or fragment thereof having at least about 85% amino acid identity to NCBI Accession No. NP_000570.1, incorporated herein by reference, and having chemokine receptor activity, as reproduced below (SEQ ID NO: 10):

| | |
|---|---|
| 1 | mdyqvsspiy dinyytsepc qkinvkqiaa rllpplyslv fifgfvgnml vililinckr |
| 61 | lksmtdiyll nlaisdlffl ltvpfwahya aaqwdfgntm cqlltglyfi gffsgiffii |
| 121 | lltidrylav vhavfalkar tvtfgvvtsv itwvvavfas lpgiiftrsq keglhytcss |
| 181 | hfpysqyqfw knfqtlkivi lglvlpllvm vicysgilkt llrcrnekkr hravrlifti |
| 241 | mivyflfwap ynivlllntf qeffglnncs ssnrldqamq vtetlgmthc cinpiiyafv |
| 301 | gekfrnyllv ffqkhiakrf ckccsifqqe aperassvyt rstgeqeisv gl |

By “C-X3-C Motif Chemokine Ligand 1 (CX3CL1) nucleic acid molecule” is meant a polynucleotide encoding a CX3CL1 polypeptide. An exemplary CX3CL1 nucleic acid molecule is provided at NCBI Accession No. NM_002996.4, incorporated herein by reference, and reproduced below (SEQ ID NO: 11):

| | |
|---|---|
| 1 | ataaaaagcc acagatctct ggcggcggca aggggacagc actgagctct gccgcctggc |
| 61 | tctagccgcc tgcctggccc ccgccgggac tcttgcccac cctcagccat ggctccgata |
| 121 | tctctgtcgt ggctgctccg cttggccacc ttctgccatc tgactgtcct gctggctgga |
| 181 | cagcaccacg gtgtgacgaa atgcaacatc acgtgcagca agatgacatc aaagatacct |
| 241 | gtagctttgc tcatccacta tcaacagaac caggcatcat gcggcaaacg cgcaatcatc |
| 301 | ttggagacga gacagcacag gctgttctgt gccgacccga aggagcaatg ggtcaaggac |
| 361 | gcgatgcagc atctggaccg ccaggctgct gccctaactc gaaatggcgg caccttcgag |
| 421 | aagcagatcg gcgaggtgaa gcccaggacc acccctgccg ccgggggaat ggacgagtct |
| 481 | gtggtcctgg agcccgaagc cacaggcgaa agcagtagcc tggagccgac tccttcttcc |
| 541 | caggaagcac agagggccct ggggacctcc ccagagctgc cgacgggcgt gactggttcc |
| 601 | tcagggacca ggctcccccc gacgccaaag gctcaggatg gagggcctgt gggcacggag |
| 661 | cttttccgag tgcctcccgt ctccactgcc gccacgtggc agagttctgc tccccaccaa |
| 721 | cctgggccca gcctctgggc tgaggcaaag acctctgagg ccccgtccac ccaggacccc |
| 781 | tccacccagg cctccactgc gtcctcccca gccccagagg agaatgctcc gtctgaaggc |
| 841 | cagcgtgtgt ggggtcaggg acagagcccc aggccagaga actctctgga gcgggaggag |
| 901 | atgggtcccg tgccagcgca cacggatgcc ttccaggact gggggcctgg cagcatggcc |
| 961 | cacgtctctg tggtccctgt ctcctcagaa gggaccccca gcagggagcc agtggcttca |
| 1021 | ggcagctgga cccctaaggc tgaggaaccc atccatgcca ccatggaccc ccagaggctg |
| 1081 | ggcgtcctta tcactcctgt ccctgacgcc caggctgcca cccggaggca ggcggtgggg |
| 1141 | ctgctggcct tccttggcct cctcttctgc ctgggggtgg ccatgttcac ctaccagagc |
| 1201 | ctccagggct gccctcgaaa gatggcagga gagatggcgg agggccttcg ctacatcccc |
| 1261 | cggagctgtg gtagtaattc atatgtcctg gtgcccgtgt gaactcctct ggcctgtgtc |
| 1321 | tagttgtttg attcagacag ctgcctggga tccctcatcc tcatacccac ccccacccaa |
| 1381 | gggcctggcc tgagctggga tgattggagg ggggaggtgg gatcctccag gtgcacaagc |
| 1441 | tccaagctcc caggcattcc ccaggaggcc agccttgacc attctccacc tgccagggac |
| 1501 | agagggggtg gcctcccaac tcaccccagc cccaaaactc tcctctgctg ctggctggtt |
| 1561 | agaggttccc tttgacgcca tcccagcccc aatgaacaat tatttattaa atgcccagcc |
| 1621 | ccttctgacc catgctgccc tgtgagtact acagtcctcc catctcacac atgagcatca |
| 1681 | ggccaggccc tctgcccact ccctgcaacc tgattgtgtc tcttggtcct gctgcagttg |
| 1741 | ccagtcaccc cggccacctg cggtgctatc tcccccagcc ccatcctctg tacagagccc |
| 1801 | acgcccccac tggtgacatg tcttttcttg catgaggcta gtgtggtgtt tcctggcact |
| 1861 | gcttccagtg aggctctgcc cttggttagg cattgtggga aggggagata agggtatctg |
| 1921 | gtgactttcc tctttggtct acactgtgct gagtctgaag gctgggttct gatcctagtt |
| 1981 | ccaccatcaa gccaccaaca tactcccatc tgtgaaagga aagagggagg taaggaatac |
| 2041 | ctgtccccct gacaacactc attgacctga ggcccttctc tccagcccct ggatgcagcc |
| 2101 | tcacagtcct taccagcaga gcaccttaga cagtccctgc caatggacta acttgtcttt |
| 2161 | ggaccctgag gcccagaggg cctgcaaggg agtgagttga tagcacagac cctgccctgt |
| 2221 | gggcccccaa atggaaatgg gcagagcaga gaccatccct gaaggccccg cccaggctta |
| 2281 | gtcactgaga cagcccgggc tctgcctccc atcacccgct aagagggagg gagggctcca |
| 2341 | gacacatgtc caagaagccc aggaaaggct ccaggagcag ccacattcct gatgcttctt |
| 2401 | cagagactcc tgcaggcagc caggccacaa gacccttgtg gtcccacccc acacacgcca |
| 2461 | gattctttcc tgaggctggg ctcccttccc acctctctca ctccttgaaa acactgttct |
| 2521 | ctgccctcca agaccttctc cttcaccttt gtccccaccg cagacaggac cagggatttc |
| 2581 | catgatgttt tccatgagtc ccctgtttgt ttctgaaagg gacgctaccc gggaaggggg |
| 2641 | ctgggacatg ggaaagggga agttgtaggc ataaagtcag gggttccctt ttttggctgc |
| 2701 | tgaaggctcg agcatgcctg gatggggctg caccggctgg cctggcccct cagggtccct |
| 2761 | ggtggcagct cacctctccc ttggattgtc cccgaccctt gccgtctacc tgaggggcct |
| 2821 | cttatgggct gggttctacc caggtgctag gaacactcct tcacagatgg gtgcttggag |
| 2881 | gaaggaaacc cagctctggt ccatagagag caagacgctg tgctgccctg cccacctggc |
| 2941 | ctctgcactc ccctgctggg tgtggcgcag catattcagg aagctcaggg cctggctcag |
| 3001 | gtggggtcac tctggcagct cagagagggt gggagtgggc ccaatgcact ttgttctggc |
| 3061 | tcttccaggc tgggagagcc ttccaggggt gggacaccct gtgatggggc cctgcctcct |
| 3121 | ttgtgaggaa gccgctgggg ccagttggtc ccccttccat ggactttgtt agtttctcca |
| 3181 | agcaggacat ggacaaggat gatctaggaa gactttggaa agagtaggaa gactttggaa |
| 3241 | agacttttcc aaccctcatc accaacgtct gtgccatttt gtattttact aataaaattt |
| 3301 | aaaagtcttg tgaatcaaaa aaaaaaaaaa aaaaaaaa |

By “CX3CL1 polypeptide” is meant a polypeptide or fragment thereof having at least about 85% amino acid identity to NCBI Accession No. NP_002987.1, incorporated herein by reference, and having chemokine activity, as reproduced below (SEQ ID NO: 12):

| | |
|---|---|
| 1 | mapislswll rlatfchltv llagqhhgvt kcnitcskmt skipvallih yqqnqascgk |
| 61 | raiiletrqh rlfcadpkeq wvkdamqhld rqaaaltrng gtfekqigev kprttpaagg |
| 121 | mdesvvlepe atgessslep tpssqeaqra lgtspelptg vtgssgtrlp ptpkaqdggp |
| 181 | vgtelfrvpp vstaatwqss aphqpgpslw aeaktseaps tqdpstqast asspapeena |
| 241 | psegqrvwgq gqsprpensl ereemgpvpa htdafqdwgp gsmahvsvvp vssegtpsre |
| 301 | pvasgswtpk aeepihatmd pqrlgvlitp vpdaqaatrr qavgllaflg llfclgvamf |
| 361 | tyqslqgcpr kmagemaegl ryiprscgsn syvlvpv |

By “Intercellular Adhesion Molecule 1 (ICAM1) nucleic acid molecule” is meant a polynucleotide encoding an ICAM1 polypeptide. An exemplary ICAM1 nucleic acid molecule is provided at NCBI Accession No. NM_000201.2, incorporated herein by reference, and reproduced below (SEQ ID NO: 13):

| | |
|---|---|
| 1 | caagcttagc ctggccggga aacgggaggc gtggaggccg ggagcagccc ccggggtcat |
| 61 | cgccctgcca ccgccgcccg attgctttag cttggaaatt ccggagctga agcggccagc |
| 121 | gagggaggat gaccctctcg gcccgggcac cctgtcagtc cggaaataac tgcagcattt |
| 181 | gttccggagg ggaaggcgcg aggtttccgg gaaagcagca ccgccccttg gcccccaggt |
| 241 | ggctagcgct ataaaggatc acgcgcccca gtcgacgctg agctcctctg ctactcagag |
| 301 | ttgcaacctc agcctcgcta tggctcccag cagcccccgg cccgcgctgc ccgcactcct |
| 361 | ggtcctgctc ggggctctgt tcccaggacc tggcaatgcc cagacatctg tgtccccctc |
| 421 | aaaagtcatc ctgccccggg gaggctccgt gctggtgaca tgcagcacct cctgtgacca |
| 481 | gcccaagttg ttgggcatag agaccccgtt gcctaaaaag gagttgctcc tgcctgggaa |
| 541 | caaccggaag gtgtatgaac tgagcaatgt gcaagaagat agccaaccaa tgtgctattc |
| 601 | aaactgccct gatgggcagt caacagctaa aaccttcctc accgtgtact ggactccaga |
| 661 | acgggtggaa ctggcacccc tcccctcttg gcagccagtg ggcaagaacc ttaccctacg |
| 721 | ctgccaggtg gagggtgggg caccccgggc caacctcacc gtggtgctgc tccgtgggga |
| 781 | gaaggagctg aaacgggagc cagctgtggg ggagcccgct gaggtcacga ccacggtgct |
| 841 | ggtgaggaga gatcaccatg gagccaattt ctcgtgccgc actgaactgg acctgcggcc |
| 901 | ccaagggctg gagctgtttg agaacacctc ggccccctac cagctccaga cctttgtcct |
| 961 | gccagcgact cccccacaac ttgtcagccc ccgggtccta gaggtggaca cgcaggggac |
| 1021 | cgtggtctgt tccctggacg ggctgttccc agtctcggag gcccaggtcc acctggcact |
| 1081 | gggggaccag aggttgaacc ccacagtcac ctatggcaac gactccttct cggccaaggc |
| 1141 | ctcagtcagt gtgaccgcag aggacgaggg cacccagcgg ctgacgtgtg cagtaatact |
| 1201 | ggggaaccag agccaggaga cactgcagac agtgaccatc tacagctttc cggcgcccaa |
| 1261 | cgtgattctg acgaagccag aggtctcaga agggaccgag gtgacagtga agtgtgaggc |
| 1321 | ccaccctaga gccaaggtga cgctgaatgg ggttccagcc cagccactgg gcccgagggc |
| 1381 | ccagctcctg ctgaaggcca ccccagagga caacgggcgc agcttctcct gctctgcaac |
| 1441 | cctggaggtg gccggccagc ttatacacaa gaaccagacc cgggagcttc gtgtcctgta |
| 1501 | tggcccccga ctggacgaga gggattgtcc gggaaactgg acgtggccag aaaattccca |
| 1561 | gcagactcca atgtgccagg cttgggggaa cccattgccc gagctcaagt gtctaaagga |
| 1621 | tggcactttc ccactgccca tcggggaatc agtgactgtc actcgagatc ttgagggcac |
| 1681 | ctacctctgt cgggccagga gcactcaagg ggaggtcacc cgcaaggtga ccgtgaatgt |
| 1741 | gctctccccc cggtatgaga ttgtcatcat cactgtggta gcagccgcag tcataatggg |
| 1801 | cactgcaggc ctcagcacgt acctctataa ccgccagcgg aagatcaaga aatacagact |
| 1861 | acaacaggcc caaaaaggga cccccatgaa accgaacaca caagccacgc ctccctgaac |
| 1921 | ctatcccggg acagggcctc ttcctcggcc ttcccatatt ggtggcagtg gtgccacact |
| 1981 | gaacagagtg gaagacatat gccatgcagc tacacctacc ggccctggga cgccggagga |
| 2041 | cagggcattg tcctcagtca gatacaacag catttggggc catggtacct gcacacctaa |
| 2101 | aacactaggc cacgcatctg atctgtagtc acatgactaa gccaagagga aggagcaaga |
| 2161 | ctcaagacat gattgatgga tgttaaagtc tagcctgatg agaggggaag tggtggggga |
| 2221 | gacatagccc caccatgagg acatacaact gggaaatact gaaacttgct gcctattggg |
| 2281 | tatgctgagg ccccacagac ttacagaaga agtggccctc catagacatg tgtagcatca |
| 2341 | aaacacaaag gcccacactt cctgacggat gccagcttgg gcactgctgt ctactgaccc |
| 2401 | caacccttga tgatatgtat ttattcattt gttattttac cagctattta ttgagtgtct |
| 2461 | tttatgtagg ctaaatgaac ataggtctct ggcctcacgg agctcccagt cctaatcaca |
| 2521 | ttcaaggtca ccaggtacag ttgtacaggt tgtacactgc aggagagtgc ctggcaaaaa |
| 2581 | gatcaaatgg ggctgggact tctcattggc caacctgcct ttccccagaa ggagtgattt |
| 2641 | ttctatcggc acaaaagcac tatatggact ggtaatggtt acaggttcag agattaccca |
| 2701 | gtgaggcctt attcctccct tccccccaaa actgacacct ttgttagcca cctccccacc |
| 2761 | cacatacatt tctgccagtg ttcacaatga cactcagcgg tcatgtctgg acatgagtgc |
| 2821 | ccagggaata tgcccaagct atgccttgtc ctcttgtcct gtttgcattt cactgggagc |
| 2881 | ttgcactatg cagctccagt ttcctgcagt gatcagggtc ctgcaagcag tggggaaggg |
| 2941 | ggccaaggta ttggaggact ccctcccagc tttggaagcc tcatccgcgt gtgtgtgtgt |
| 3001 | gtgtatgtgt agacaagctc tcgctctgtc acccaggctg gagtgcagtg gtgcaatcat |
| 3061 | ggttcactgc agtcttgacc ttttgggctc aagtgatcct cccacctcag cctcctgagt |
| 3121 | agctgggacc ataggctcac aacaccacac ctggcaaatt tgattttttt tttttttcca |
| 3181 | gagacggggt ctcgcaacat tgcccagact tcctttgtgt tagttaataa agctttctca |
| 3241 | actgccaaa |

By “ICAM1 polypeptide” is meant a polypeptide or fragment thereof having at least about 85% amino acid identity to NCBI Accession No. NP_000192.2, incorporated herein by reference, and having ICAM1 activity, as reproduced below (SEQ ID NO: 14):

| | |
|---|---|
| 1 | mapssprpal pallvllgal fpgpgnaqts vspskvilpr ggsvlvtcst scdqpkllgi |
| 61 | etplpkkell lpgnnrkvye lsnvqedsqp mcysncpdgq staktfltvy wtpervelap |
| 121 | lpswqpvgkn ltlrcqvegg apranltvvl lrgekelkre pavgepaevt ttvlvrrdhh |
| 181 | ganfscrtel dlrpqglelf entsapyqlq tfvlpatppq lvsprvlevd tqgtvvcsld |
| 241 | glfpvseaqv hlalgdqrln ptvtygndsf sakasvsvta edegtqrltc avilgnqsqe |
| 301 | tlqtvtiysf papnviltkp evsegtevtv kceahprakv tlngvpaqpl gpraqlllka |
| 361 | tpedngrsfs csatlevagq lihknqtrel rvlygprlde rdcpgnwtwp ensqqtpmcq |
| 421 | awgnplpelk clkdgtfplp igesvtvtrd legtylcrar stqgevtrkv tvnvlsprye |
| 481 | iviitvvaaa vimgtaglst ylynrqrkik kyrlqqaqkg tpmkpntqat pp |

By “Vascular Cell Adhesion Molecule 1 (VCAM1) nucleic acid molecule” is meant a polynucleotide encoding a VCAM1 polypeptide. An exemplary VCAM1 nucleic acid molecule is provided at NCBI Accession No. NM_001078.3, incorporated herein by reference, and reproduced below (SEQ ID NO: 15):

| | |
|---|---|
| 1 | aaactttttt ccctggctct gccctgggtt tccccttgaa gggatttccc tccgcctctg |
| 61 | caacaagacc ctttataaag cacagacttt ctatttcact ccgcggtatc tgcatcgggc |
| 121 | ctcactggct tcaggagctg aataccctcc caggcacaca caggtgggac acaaataagg |
| 181 | gttttggaac cactattttc tcatcacgac agcaacttaa aatgcctggg aagatggtcg |
| 241 | tgatccttgg agcctcaaat atactttgga taatgtttgc agcttctcaa gcttttaaaa |
| 301 | tcgagaccac cccagaatct agatatcttg ctcagattgg tgactccgtc tcattgactt |
| 361 | gcagcaccac aggctgtgag tccccatttt tctcttggag aacccagata gatagtccac |
| 421 | tgaatgggaa ggtgacgaat gaggggacca catctacgct gacaatgaat cctgttagtt |
| 481 | ttgggaacga acactcttac ctgtgcacag caacttgtga atctaggaaa ttggaaaaag |
| 541 | gaatccaggt ggagatctac tcttttccta aggatccaga gattcatttg agtggccctc |
| 601 | tggaggctgg gaagccgatc acagtcaagt gttcagttgc tgatgtatac ccatttgaca |
| 661 | ggctggagat agacttactg aaaggagatc atctcatgaa gagtcaggaa tttctggagg |
| 721 | atgcagacag gaagtccctg gaaaccaaga gtttggaagt aacctttact cctgtcattg |
| 781 | aggatattgg aaaagttctt gtttgccgag ctaaattaca cattgatgaa atggattctg |
| 841 | tgcccacagt aaggcaggct gtaaaagaat tgcaagtcta catatcaccc aagaatacag |
| 901 | ttatttctgt gaatccatcc acaaagctgc aagaaggtgg ctctgtgacc atgacctgtt |
| 961 | ccagcgaggg tctaccagct ccagagattt tctggagtaa gaaattagat aatgggaatc |
| 1021 | tacagcacct ttctggaaat gcaactctca ccttaattgc tatgaggatg gaagattctg |
| 1081 | gaatttatgt gtgtgaagga gttaatttga ttgggaaaaa cagaaaagag gtggaattaa |
| 1141 | ttgttcaaga gaaaccattt actgttgaga tctcccctgg accccggatt gctgctcaga |
| 1201 | ttggagactc agtcatgttg acatgtagtg tcatgggctg tgaatcccca tctttctcct |
| 1261 | ggagaaccca gatagacagc cctctgagcg ggaaggtgag gagtgagggg accaattcca |
| 1321 | cgctgaccct gagccctgtg agttttgaga acgaacactc ttatctgtgc acagtgactt |
| 1381 | gtggacataa gaaactggaa aagggaatcc aggtggagct ctactcattc cctagagatc |
| 1441 | cagaaatcga gatgagtggt ggcctcgtga atgggagctc tgtcactgta agctgcaagg |
| 1501 | ttcctagcgt gtaccccctt gaccggctgg agattgaatt acttaagggg gagactattc |
| 1561 | tggagaatat agagtttttg gaggatacgg atatgaaatc tctagagaac aaaagtttgg |
| 1621 | aaatgacctt catccctacc attgaagata ctggaaaagc tcttgtttgt caggctaagt |
| 1681 | tacatattga tgacatggaa ttcgaaccca aacaaaggca gagtacgcaa acactttatg |
| 1741 | tcaatgttgc ccccagagat acaaccgtct tggtcagccc ttcctccatc ctggaggaag |
| 1801 | gcagttctgt gaatatgaca tgcttgagcc agggctttcc tgctccgaaa atcctgtgga |
| 1861 | gcaggcagct ccctaacggg gagctacagc ctctttctga gaatgcaact ctcaccttaa |
| 1921 | tttctacaaa aatggaagat tctggggttt atttatgtga aggaattaac caggctggaa |
| 1981 | gaagcagaaa ggaagtggaa ttaattatcc aagttactcc aaaagacata aaacttacag |
| 2041 | cttttccttc tgagagtgtc aaagaaggag acactgtcat catctcttgt acatgtggaa |
| 2101 | atgttccaga aacatggata atcctgaaga aaaaagcgga gacaggagac acagtactaa |
| 2161 | aatctataga tggcgcctat accatccgaa aggcccagtt gaaggatgcg ggagtatatg |
| 2221 | aatgtgaatc taaaaacaaa gttggctcac aattaagaag tttaacactt gatgttcaag |
| 2281 | gaagagaaaa caacaaagac tatttttctc ctgagcttct cgtgctctat tttgcatcct |
| 2341 | ccttaataat acctgccatt ggaatgataa tttactttgc aagaaaagcc aacatgaagg |
| 2401 | ggtcatatag tcttgtagaa gcacagaagt caaaagtgta gctaatgctt gatatgttca |
| 2461 | actggagaca ctatttatct gtgcaaatcc ttgatactgc tcatcattcc ttgagaaaaa |
| 2521 | caatgagctg agaggcagac ttccctgaat gtattgaact tggaaagaaa tgcccatcta |
| 2581 | tgtcccttgc tgtgagcaag aagtcaaagt aaaacttgct gcctgaagaa cagtaactgc |
| 2641 | catcaagatg agagaactgg aggagttcct tgatctgtat atacaataac ataatttgta |
| 2701 | catatgtaaa ataaaattat gccatagcaa gattgcttaa aatagcaaca ctctatattt |
| 2761 | agattgttaa aataactagt gttgcttgga ctattataat ttaatgcatg ttaggaaaat |
| 2821 | ttcacattaa tatttgctga cagctgacct ttgtcatctt tcttctattt tattcccttt |
| 2881 | cacaaaattt tattcctata tagtttattg acaataattt caggttttgt aaagatgccg |
| 2941 | ggttttatat ttttatagac aaataataag caaagggagc actgggttga ctttcaggta |
| 3001 | ctaaatacct caacctatgg tataatggtt gactgggttt ctctgtatag tactggcatg |
| 3061 | gtacggagat gtttcacgaa gtttgttcat cagactcctg tgcaactttc ccaatgtggc |
| 3121 | ctaaaaatgc aacttctttt tattttcttt tgtaaatgtt taggtttttt tgtatagtaa |
| 3181 | agtgataatt tctggaatta gaaaaaaaaa aaaaaaaaaa |

By ““Vascular Cell Adhesion Molecule 1 (VCAM1)” is meant a polypeptide or fragment thereof having at least about 85% amino acid identity to NCBI Accession No. NP_001069.1, incorporated herein by reference, and having chemokine activity, as reproduced below (SEQ ID NO: 16):

| | |
|---|---|
| 1 | mpgkmvvilg asnilwimfa asqafkiett pesrylaqig dsvsltcstt gcespffswr |
| 61 | tqidsplngk vtnegttstl tmnpvsfgne hsylctatce srklekgiqv eiysfpkdpe |
| 121 | ihlsgpleag kpitvkcsva dvypfdrlei dllkgdhlmk sqefledadr ksletkslev |
| 181 | tftpviedig kvlvcraklh idemdsvptv rqavkelqvy ispkntvisv npstklqegg |
| 241 | svtmtcsseg lpapeifwsk kldngnlqhl sgnatltlia mrmedsgiyv cegvnligkn |
| 301 | rkevelivqe kpftveispg priaaqigds vmltcsvmgc espsfswrtq idsplsgkvr |
| 361 | segtnstltl spvsfenehs ylctvtcghk klekgiqvel ysfprdpeie msgglvngss |
| 421 | vtvsckvpsv ypldrleiel lkgetileni efledtdmks lenkslemtf iptiedtgka |
| 481 | lvcqaklhid dmefepkqrq stqtlyvnva prdttvlvsp ssileegssv nmtclsqgfp |
| 541 | apkilwsrql pngelqplse natltlistk medsgvylce ginqagrsrk eveliiqvtp |
| 601 | kdikltafps esvkegdtvi isctcgnvpe twiilkkkae tgdtvlksid gaytirkaql |
| 661 | kdagvyeces knkvgsqlrs ltldvqgren nkdyfspell vlyfasslii paigmiiyfa |
| 721 | rkanmkgsys lveaqkskv |

By “CD47 Molecule (CD47) nucleic acid molecule” is meant a polynucleotide encoding a CD47 polypeptide. An exemplary CD47 nucleic acid molecule is provided at NCBI Accession No. NM_001777.3, incorporated herein by reference, and reproduced below (SEQ ID NO: 17):

| | |
|---|---|
| 1 | ggggagcagg cgggggagcg ggcgggaagc agtgggagcg cgcgtgcgcg cggccgtgca |
| 61 | gcctgggcag tgggtcctgc ctgtgacgcg cggcggcggt cggtcctgcc tgtaacggcg |
| 121 | gcggcggctg ctgctccaga cacctgcggc ggcggcggcg accccgcggc gggcgcggag |
| 181 | atgtggcccc tggtagcggc gctgttgctg ggctcggcgt gctgcggatc agctcagcta |
| 241 | ctatttaata aaacaaaatc tgtagaattc acgttttgta atgacactgt cgtcattcca |
| 301 | tgctttgtta ctaatatgga ggcacaaaac actactgaag tatacgtaaa gtggaaattt |
| 361 | aaaggaagag atatttacac ctttgatgga gctctaaaca agtccactgt ccccactgac |
| 421 | tttagtagtg caaaaattga agtctcacaa ttactaaaag gagatgcctc tttgaagatg |
| 481 | gataagagtg atgctgtctc acacacagga aactacactt gtgaagtaac agaattaacc |
| 541 | agagaaggtg aaacgatcat cgagctaaaa tatcgtgttg tttcatggtt ttctccaaat |
| 601 | gaaaatattc ttattgttat tttcccaatt tttgctatac tcctgttctg gggacagttt |
| 661 | ggtattaaaa cacttaaata tagatccggt ggtatggatg agaaaacaat tgctttactt |
| 721 | gttgctggac tagtgatcac tgtcattgtc attgttggag ccattctttt cgtcccaggt |
| 781 | gaatattcat taaagaatgc tactggcctt ggtttaattg tgacttctac agggatatta |
| 841 | atattacttc actactatgt gtttagtaca gcgattggat taacctcctt cgtcattgcc |
| 901 | atattggtta ttcaggtgat agcctatatc ctcgctgtgg ttggactgag tctctgtatt |
| 961 | gcggcgtgta taccaatgca tggccctctt ctgatttcag gtttgagtat cttagctcta |
| 1021 | gcacaattac ttggactagt ttatatgaaa tttgtggctt ccaatcagaa gactatacaa |
| 1081 | cctcctagga aagctgtaga ggaacccctt aatgcattca aagaatcaaa aggaatgatg |
| 1141 | aatgatgaat aactgaagtg aagtgatgga ctccgatttg gagagtagta agacgtgaaa |
| 1201 | ggaatacact tgtgtttaag caccatggcc ttgatgattc actgttgggg agaagaaaca |
| 1261 | agaaaagtaa ctggttgtca cctatgagac ccttacgtga ttgttagtta agtttttatt |
| 1321 | caaagcagct gtaatttagt taataaaata attatgatct atgttgtttg cccaattgag |
| 1381 | atccagtttt ttgttgttat ttttaatcaa ttaggggcaa tagtagaatg gacaatttcc |
| 1441 | aagaatgatg cctttcaggt cctagggcct ctggcctcta ggtaaccagt ttaaattggt |
| 1501 | tcagggtgat aactacttag cactgccctg gtgattaccc agagatatct atgaaaacca |
| 1561 | gtggcttcca tcaaaccttt gccaactcag gttcacagca gctttgggca gttatggcag |
| 1621 | tatggcatta gctgagaggt gtctgccact tctgggtcaa tggaataata aattaagtac |
| 1681 | aggcaggaat ttggttggga gcatcttgta tgatctccgt atgatgtgat attgatggag |
| 1741 | atagtggtcc tcattcttgg gggttgccat tcccacattc ccccttcaac aaacagtgta |
| 1801 | acaggtcctt cccagattta gggtactttt attgatggat atgttttcct tttattcaca |
| 1861 | taaccccttg aaaccctgtc ttgtcctcct gttacttgct tctgctgtac aagatgtagc |
| 1921 | accttttctc ctctttgaac atggtctagt gacacggtag caccagttgc aggaaggagc |
| 1981 | cagacttgtt ctcagagcac tgtgttcaca cttttcagca aaaatagcta tggttgtaac |
| 2041 | atatgtattc ccttcctctg atttgaaggc aaaaatctac agtgtttctt cacttctttt |
| 2101 | ctgatctggg gcatgaaaaa agcaagattg aaatttgaac tatgagtctc ctgcatggca |
| 2161 | acaaaatgtg tgtcaccatc aggccaacag gccagccctt gaatggggat ttattactgt |
| 2221 | tgtatctatg ttgcatgata aacattcatc accttcctcc tgtagtcctg cctcgtactc |
| 2281 | cccttcccct atgattgaaa agtaaacaaa acccacattt cctatcctgg ttagaagaaa |
| 2341 | attaatgttc tgacagttgt gatcgcctgg agtactttta gacttttagc attcgttttt |
| 2401 | tacctgtttg tggatgtgtg tttgtatgtg catacgtatg agataggcac atgcatcttc |
| 2461 | tgtatggaca aaggtggggt acctacagga gagcaaaggt taattttgtg cttttagtaa |
| 2521 | aaacatttaa atacaaagtt ctttattggg tggaattata tttgatgcaa atatttgatc |
| 2581 | acttaaaact tttaaaactt ctaggtaatt tgccacgctt tttgactgct caccaatacc |
| 2641 | ctgtaaaaat acgtaattct tcctgtttgt gtaataagat attcatattt gtagttgcat |
| 2701 | taataatagt tatttcttag tccatcagat gttcccgtgt gcctctttta tgccaaattg |
| 2761 | attgtcatat ttcatgttgg gaccaagtag tttgcccatg gcaaacctaa atttatgacc |
| 2821 | tgctgaggcc tctcagaaaa ctgagcatac tagcaagaca gctcttcttg aaaaaaaaaa |
| 2881 | tatgtataca caaatatata cgtatatcta tatatacgta tgtatataca cacatgtata |
| 2941 | ttcttccttg attgtgtagc tgtccaaaat aataacatat atagagggag ctgtattcct |
| 3001 | ttatacaaat ctgatggctc ctgcagcact ttttccttct gaaaatattt acattttgct |
| 3061 | aacctagttt gttactttaa aaatcagttt tgatgaaagg agggaaaagc agatggactt |
| 3121 | gaaaaagatc caagctccta ttagaaaagg tatgaaaatc tttatagtaa aattttttat |
| 3181 | aaactaaagt tgtacctttt aatatgtagt aaactctcat ttatttgggg ttcgctcttg |
| 3241 | gatctcatcc atccattgtg ttctctttaa tgctgcctgc cttttgaggc attcactgcc |
| 3301 | ctagacaatg ccaccagaga tagtggggga aatgccagat gaaaccaact cttgctctca |
| 3361 | ctagttgtca gcttctctgg ataagtgacc acagaagcag gagtcctcct gcttgggcat |
| 3421 | cattgggcca gttccttctc tttaaatcag atttgtaatg gctcccaaat tccatcacat |
| 3481 | cacatttaaa ttgcagacag tgttttgcac atcatgtatc tgttttgtcc cataatatgc |
| 3541 | tttttactcc ctgatcccag tttctgctgt tgactcttcc attcagtttt atttattgtg |
| 3601 | tgttctcaca gtgacaccat ttgtcctttt ctgcaacaac ctttccagct acttttgcca |
| 3661 | aattctattt gtcttctcct tcaaaacatt ctcctttgca gttcctcttc atctgtgtag |
| 3721 | ctgctctttt gtctcttaac ttaccattcc tatagtactt tatgcatctc tgcttagttc |
| 3781 | tattagtttt ttggccttgc tcttctcctt gattttaaaa ttccttctat agctagagct |
| 3841 | tttctttctt tcattctctc ttcctgcagt gttttgcata catcagaagc taggtacata |
| 3901 | agttaaatga ttgagagttg gctgtattta gatttatcac tttttaatag ggtgagcttg |
| 3961 | agagttttct ttctttctgt tttttttttt tgtttttttt tttttttttt tttttttttt |
| 4021 | ttttgactaa tttcacatgc tctaaaaacc ttcaaaggtg attatttttc tcctggaaac |
| 4081 | tccaggtcca ttctgtttaa atccctaaga atgtcagaat taaaataaca gggctatccc |
| 4141 | gtaattggaa atatttcttt tttcaggatg ctatagtcaa tttagtaagt gaccaccaaa |
| 4201 | ttgttatttg cactaacaaa gctcaaaaca cgataagttt actcctccat ctcagtaata |
| 4261 | aaaattaagc tgtaatcaac cttctaggtt tctcttgtct taaaatgggt attcaaaaat |
| 4321 | ggggatctgt ggtgtatgta tggaaacaca tactccttaa tttacctgtt gttggaaact |
| 4381 | ggagaaatga ttgtcgggca accgtttatt ttttattgta ttttatttgg ttgagggatt |
| 4441 | tttttataaa cagttttact tgtgtcatat tttaaaatta ctaactgcca tcacctgctg |
| 4501 | gggtcctttg ttaggtcatt ttcagtgact aatagggata atccaggtaa ctttgaagag |
| 4561 | atgagcagtg agtgaccagg cagtttttct gcctttagct ttgacagttc ttaattaaga |
| 4621 | tcattgaaga ccagctttct cataaatttc tctttttgaa aaaaagaaag catttgtact |
| 4681 | aagctcctct gtaagacaac atcttaaatc ttaaaagtgt tgttatcatg actggtgaga |
| 4741 | gaagaaaaca ttttgttttt attaaatgga gcattattta caaaaagcca ttgttgagaa |
| 4801 | ttagatccca catcgtataa atatctatta accattctaa ataaagagaa ctccagtgtt |
| 4861 | gctatgtgca agatcctctc ttggagcttt tttgcatagc aattaaaggt gtgctatttg |
| 4921 | tcagtagcca tttttttgca gtgatttgaa gaccaaagtt gttttacagc tgtgttaccg |
| 4981 | ttaaaggttt ttttttttat atgtattaaa tcaatttatc actgtttaaa gctttgaata |
| 5041 | tctgcaatct ttgccaaggt acttttttat ttaaaaaaaa acataacttt gtaaatatta |
| 5101 | ccctgtaata ttatatatac ttaataaaac attttaagct attttgttgg gctatttcta |
| 5161 | ttgctgctac agcagaccac aagcacattt ctgaaaaatt taatttatta atgtattttt |
| 5221 | aagttgctta tattctaggt aacaatgtaa agaatgattt aaaatattaa ttatgaattt |
| 5281 | tttgagtata atacccaata agcttttaat tagagcagag ttttaattaa aagttttaaa |
| 5341 | tcagtc |

By “CD47 polypeptide” is meant a polypeptide or fragment thereof having at least about 85% amino acid identity to NCBI Accession No. NP_001768.1, incorporated herein by reference, and having binding activity, as reproduced below (SEQ ID NO: 18):

| | |
|---|---|
| 1 | mwplvaalll gsaccgsaql lfnktksvef tfcndtvvip cfvtnmeaqn ttevyvkwkf |
| 61 | kgrdiytfdg alnkstvptd fssakievsq llkgdaslkm dksdavshtg nytcevtelt |
| 121 | regetiielk yrvvswfspn enilivifpi faillfwgqf giktlkyrsg gmdektiall |
| 181 | vaglvitviv ivgailfvpg eyslknatgl glivtstgil illhyyvfst aigltsfvia |
| 241 | ilviqviayi lavvglslci aacipmhgpl lisglsilal aqllglvymk fvasnqktiq |
| 301 | pprkaveepl nafkeskgmm nde |

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia, e.g., the leukemia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

The administration of a compound or a combination of compounds for the treatment of a neoplasia, e.g., a leukemia, may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia (e.g., leukemia). Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### EXAMPLES

The present invention is further illustrated by the following examples, which should not be construed as limiting. The contents of all references, GenBank Accession and Gene numbers, and published patents and patent applications cited throughout the application are hereby incorporated by reference. Those skilled in the art will recognize that the invention may be practiced with variations on the described structures, materials, compositions and methods, and such variations are regarded as within the scope of the invention.

### Example 1: CTLA4 Blockade After Allogenic Transplantation

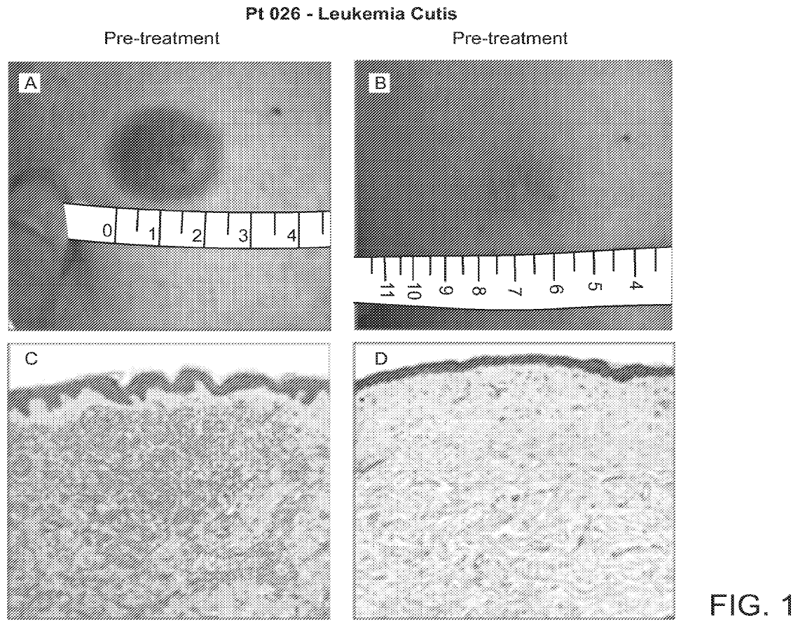

CTLA4 inhibition may re-awaken a dormant Graft-versus-leukemia (GVL) effect with less toxicity than donor lymphocyte infusions (DLI). To assess whether CTLA4 blockade is safe and/or effective after allogenic transplantation, 28 patients with relapsed hematologic cancer were treated with Ipilimumab after allogenic hematopoietic stem cell transplantation (HSCT) at either 3 mg/kg (n=6) or 10 mg/kg (n=22). No responses were observed at the low-dose; however, 59% of the high-dose patients had tumor reduction (including durable complete remissions) as shown in FIG. 1, which shows photographic images of a leukemia cutis associated cutaneous lesion before (top left panel) and after (top right panel) treatment with a single dose of Ipilimumab, as well as histologically stained tissue sections of the cutaneous lesion before (bottom left panel) and after (bottom right panel) treatment.

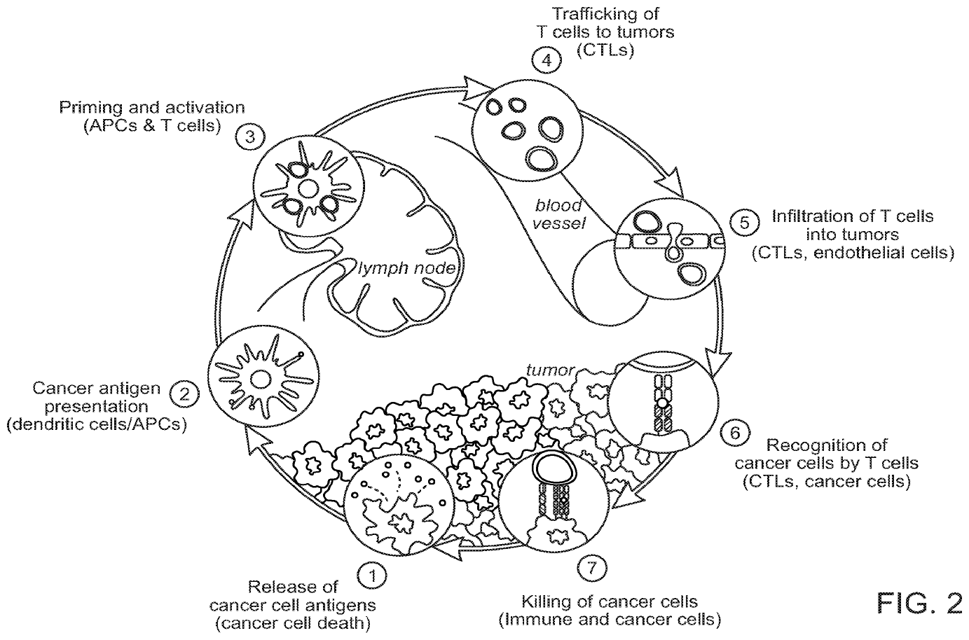

FIG. 2 depicts a schematic of the Cancer-Immunity Cycle, which is a theoretical construct that describes the iterative steps that occur during the generation of an effective anti-cancer immune response.

To identify immune-related genes relevant for predict response to Ipilimumab, 11 tumor biopsies from four patients were subjected to RNA sequencing. Two of the patients had a complete response to Ipilimumab, and one of these two patients had two tumor sites so the total sample included 6 tumors from 3 sites pre/post Ipilimumab from the responding patients. One of the patients had a transient response followed by relapse, thus there were three samples from this patient. One of the four patients did not respond to Ipilimumab at all, and there were two samples (pre/post) from this patient. As described further below, these studies made it possible to infer immune subpopulations and activation states in leukemic microenvironment (LME).

### Example 2: Role of Ipilimumab in Immune Cell Trafficking

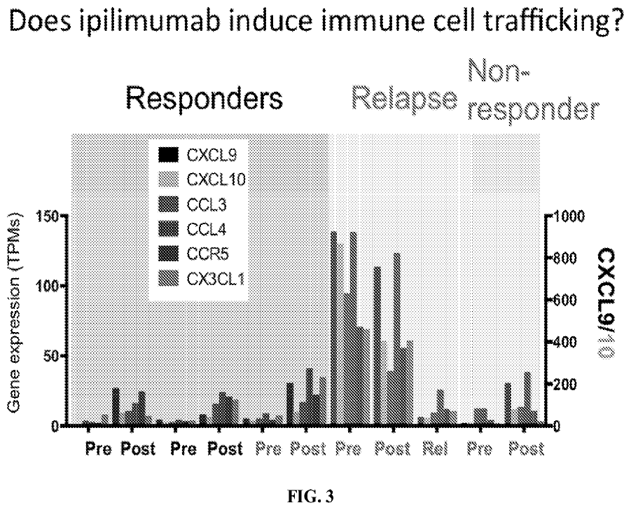

As shown in FIG. 3, there was an increase in expression of genes responsible for immune cell trafficking after Ipilimumab treatment in both responding and nonresponding tumors as indicated by the difference in expression levels pre- and post-treatment. Expression levels were tested for the following genes: C-X-C Motif Chemokine Ligand 9 (CXCL9; shown in black), C-X-C Motif Chemokine Ligand 10 (CXCL10; shown in orange), C-C Motif Chemokine Ligand 3 (CCL3; shown in maroon), C-C Motif Chemokine Ligand 4 (CCL4; shown in purple), C-C Motif Chemokine Receptor 5 (CCR5; shown in blue), and C-X3-C Motif Chemokine Ligand 1 (CX3CL1; shown in green). As can be seen in the bar graph, pre-Ipilimumab tumor in the relapsing patient has a very high ‘inflamed’ baseline of chemokines that is downregulated in the post-Ipilimumab relapsed tumor.

### Example 3: Analysis of Leukemic Bed Infiltration

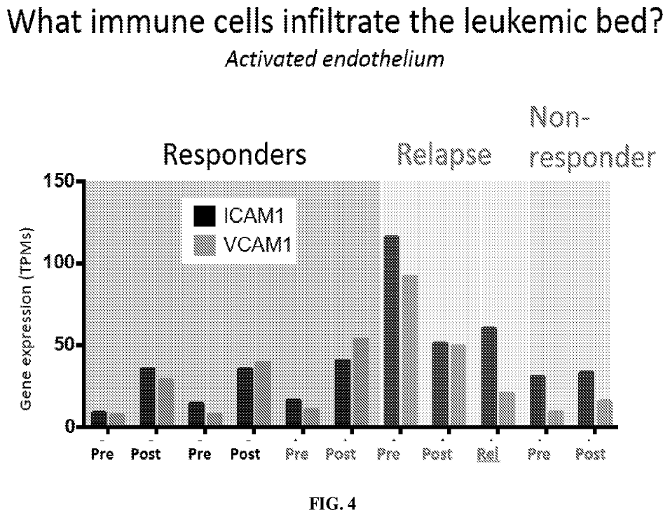

As shown in FIG. 4, there was an increase in genes responsible for immune cell trafficking through the vascular endothelium after Ipilimumab treatment in both responding and nonresponding tumors. Expression levels were tested for the following genes: Intercellular Adhesion Molecule 1 (ICAM1; shown in black) and Vascular Cell Adhesion Molecule 1 (VCAM1; shown in gray). As can be seen in FIG. 4, pre-Ipilimumab tumor in the relapsing patient has a very high ‘inflamed’ baseline of chemokines that is downregulated in the post-Ipilimumab relapsed tumor.

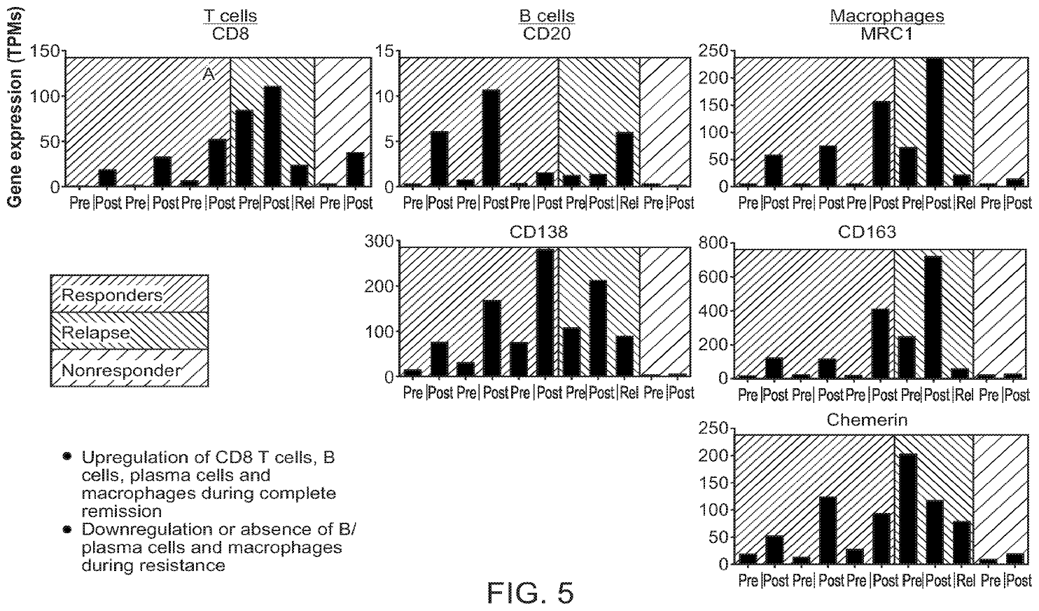

FIG. 5 assess the specific immune cell sub-populations corresponding to T cells, B cells, and Macrophages, respectively. After treatment with Ipilimumab, CD8A expression is increased in T cells, CD20 and CD138 expression is increased in B cells and plasma cells, respectively, and the expression of MRC1 and CD163 and Chemerin is increased in macrophages. However, in the non-responding patient increased CD8+ expression (blue bars) is only seen in T Cells after treatment with Ipilimumab. In the case of a relapse, all of the cell types (i.e., T cells, B cells, and Macrophages) are down-regulated (red bars).

### Example 4: Analysis of Macrophage Evasion

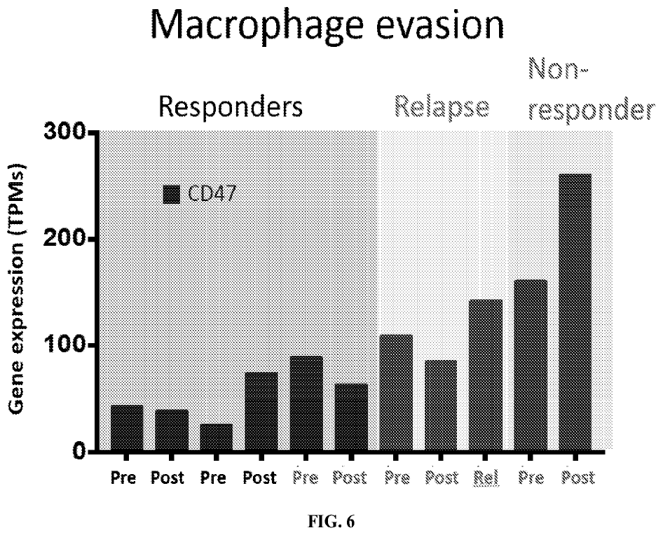

Macrophage evasion in responder, relapse, and non-responder patients, respectively, was assessed by analyzing CD47 expression to assess macrophage defense capabilities (FIG. 6). As shown, the three resistant tumors (“Rel,” “Pre,” and “Post from the non-responder) are the ones with the highest levels of CD47 expression, and they are also the ones with the lowest macrophages gene expression. Notably, CD47 expression was highest in the non-responders, indicating that CD47 may be a useful biomarker for patients resistant to treatment with CTLA4 antagonists such as, for example, Ipilimumab.

### Example 5: Leukemic Recognition by T Cells

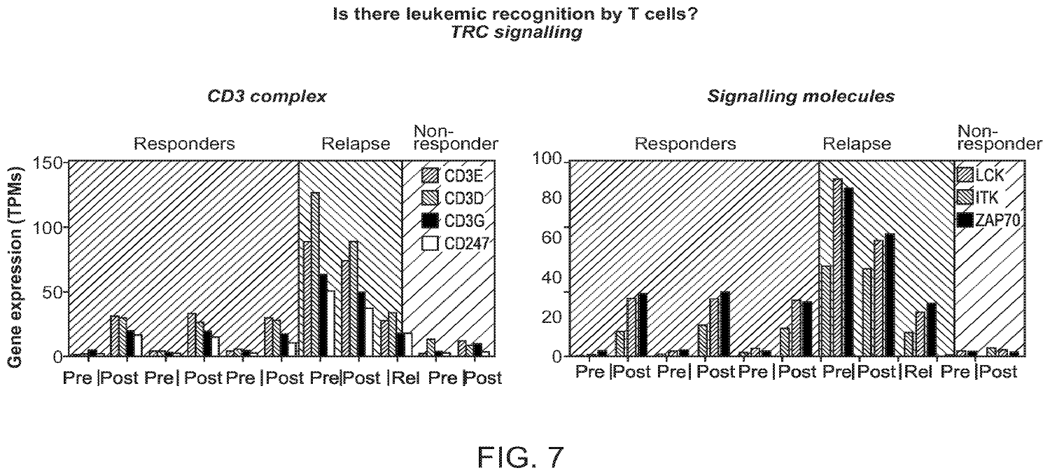

As shown in FIG. 7, leukemic recognition by T cells was assessed via expression analysis of both T cell receptor genes and T cell signaling genes. As shown in the left graph, responders are associated with T cells that express T cell receptor genes such as CD3E, CD3D, CD3G, and CD247, while these genes are not expressed in the relapse or non-responders. Similarly, responders are associated with T cells that express signaling genes such as LCK, ITK, and ZAP70, while these genes are not expressed in the relapse or non-responders (right graph). The relapse category is associated with downregulation of both the T cell receptor genes and the signaling genes.

### Example 6: Activation of In Situ T Cells

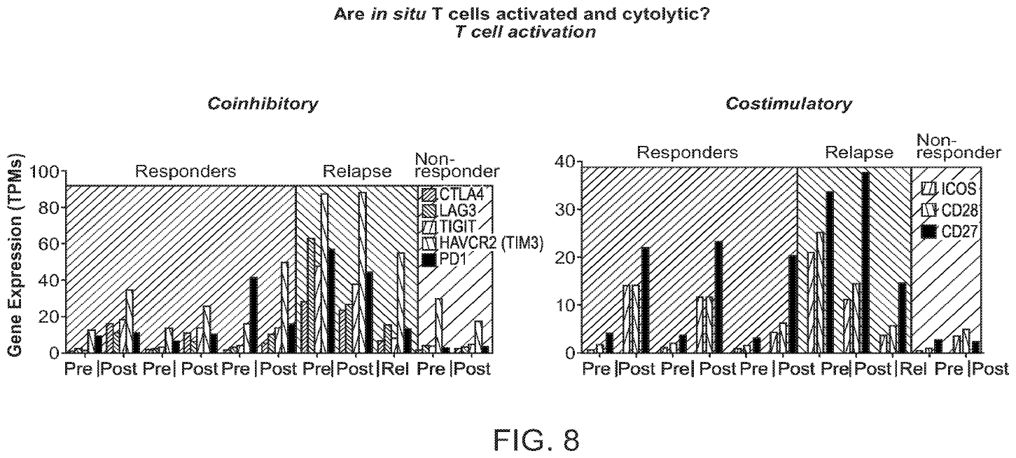

FIG. 8 shows data assessing T cell activation. As shown in the left graph, responders are associated with T cells that express coinhibitory receptor genes such as CTLA4, LAGS, TIGIT, HAVCR2, and PD1, while these genes are not expressed in the relapse or non-responders. Similarly, responders are associated with T cells that express costimulatory receptor genes such as ICOS, CD28, and CD27, while these genes are not expressed in the relapse or non-responders (right graph).

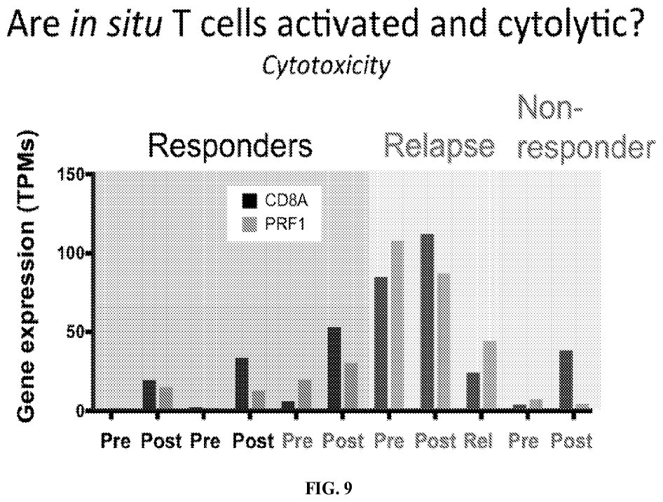

Additionally, FIG. 9 shows data assessing whether T cells are activated and cytolytic. Responders are associated with T cells that express CD8A and perforin (PRF1), indicating that their tumors are being infiltrated by CD8 T cells that are cytolytic and have the capacity to kill. Although CD8 T cells come to the tumor in the nonresponding patient after Ipilimumab treatment, these T cells are not cytolytic, as evidenced by lack of PRF1 gene expression.

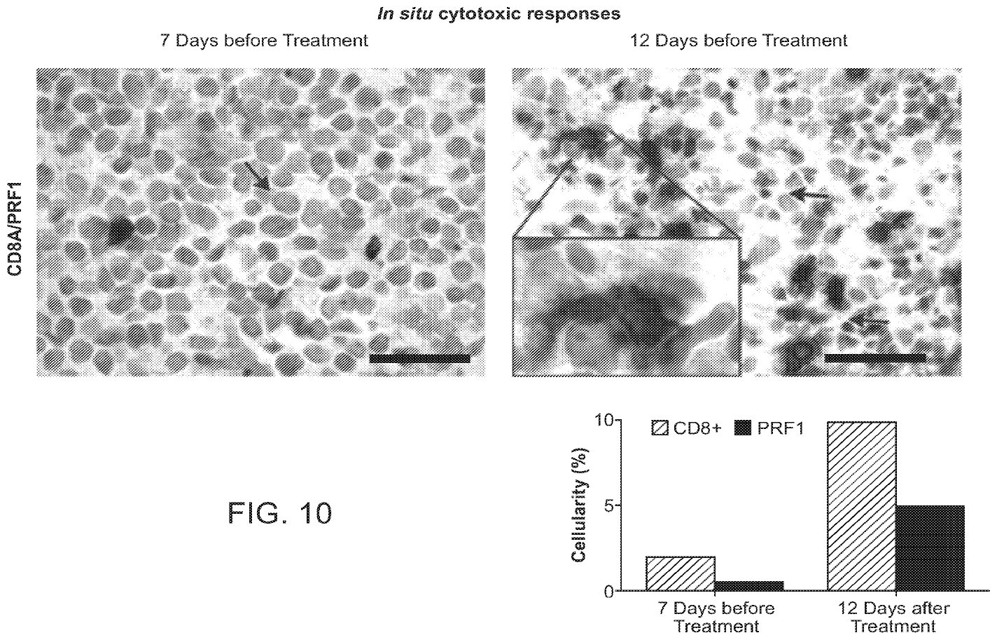

The in situ cytotoxic responses were assessed by immunohistochemistry. FIG. 10 depicts immunohistochemistry staining seven days before (left) and twelve days after (right) treatment with Ipilimumab in a responder patient. The staining data shows that CD8 T cells come into the tumor after Ipilimumab treatment (e.g., CD8A staining) and contacts and kills multiple tumor cells (e.g., PRF1 staining).

In summary, three patterns of immunologic responses were observed. First, populations that showed complete response were associated with diverse infiltration of activated CTLs, B cells, and macrophages, and also showed downregulation of immune inhibitory molecules (e.g., CD47). Second, populations that showed resistance to CTLA4 antagonists (e.g., Ipilimumab) were associated with marked upregulation of CD8 without TCR signaling, activation or cytolytic activity in addition to lack of macrophage/plasma cell infiltration and increased CD47. Third, populations that showed transient response/relapse were associated with a baseline immunologically ‘primed’ state that is downregulated]], as evidence by decreased trafficking cytokines (e.g. CXCL9, CXCL10, CCR3, CCR4, CCL5, CX3CL1), decreased activated endothelium (e.g. VCAM1, ICAM1), decreased immune subsets (e.g. CD8A, CD138, MRC1, CD163, Chemerin), decreased TCR signaling (e.g. CD3E, CD3D, CD3G, CD247; e.g. LCK, ITK, ZAP70), decreased coinhibitory/costimulatory molecules (e.g. CTLA4, LAG3, TIM3, TIGIT, PD1; e.g. ICOS, CD28, CD27), and decreased cytolysis (e.g. PRF1, GZMA). According to the techniques herein, the pretreatment immunologic state may dictate the clinical outcome to immunotherapeutic intervention in the post-allogeneic setting.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### Equivalents

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method of determining whether treatment of a subject having a leukemia with a cytotoxic T-lymphocyte-associated protein 4 (CTLA4) antagonist will result in clinical benefit to the subject, comprising:
obtaining a test sample from the subject having the leukemia;
determining the expression level of at least one leukemia-associated gene in the test sample;
comparing the expression level of the leukemia-associated gene in the test sample with the expression level of the leukemia-associated gene in a reference sample; and
determining whether the CTLA4 antagonist will inhibit leukemia in the subject if the expression level of the leukemia-associated gene in the test sample is differentially expressed relative to the level of the leukemia-associated gene in the reference sample.

2. The method of claim 1, wherein the test sample is obtained from a leukemia tissue, a tumor microenvironment, or a tumor-infiltrating immune cell.

3. The method of claim 1, wherein clinical benefit in the subject comprises complete or partial response as defined by response evaluation criteria in solid tumors (RECIST), stable disease as defined by RECIST, or long-term survival in spite of disease progression or response as defined by irRC criteria.

4. The method of claim 1, wherein the test sample is obtained from the leukemia, wherein the leukemia-associated gene comprises a CD47 molecule (CD47) gene; and
determining that treatment of the subject with leukemia with the CTLA4 antagonist will not result in clinical benefit in the subject if the expression level of the CD47 gene in the test sample is higher than the level of the CD47 gene in the reference sample.

5. The method of claim 1, wherein the test sample is obtained from the leukemia, wherein the leukemia-associated gene comprises a CD47 molecule (CD47) gene; and
determining that treatment of the subject with leukemia with the CTLA4 antagonist will result in clinical benefit in the subject if the expression level of the CD47 gene in the test sample is equal to, or lower than, the level of the CD47 gene in the reference sample.

6. The method of claim 1, wherein the sample comprises deoxyribonucleic acid (DNA) or ribonucleic acid (RNA).

7. The method of claim 1, wherein the sample comprises a plasma sample, a blood sample, a marrow sample, a lymph node, or any site of leukemic infection.

8. The method of claim 1, wherein said sample comprises circulating tumor cells.

9. The method of claim 1, wherein the reference sample is obtained from healthy normal tissue, leukemia that received a clinical benefit from CTLA4 antagonist, or leukemia that did not receive a clinical benefit from CTLA4 antagonist.

10. The method of claim 1, wherein the expression level of the leukemia-associated gene is detected via an Affymetrix Gene Array hybridization, next generation sequencing, ribonucleic acid sequencing (RNA-seq), a real time reverse transcriptase polymerase chain reaction (real time RT-PCR) assay, immunohistochemistry (IHC), immunofluorescence, or methylation-specific PCR.

11. The method of claim 1, wherein the expression level of the leukemia-associated gene is detected via RNA-seq and the reference sample is obtained from healthy normal tissue from the same individual as the test sample or one or more healthy normal tissues from different individuals.

12. The method of claim 1, wherein the expression level of the leukemia-associated gene is detected via RT-PCR and wherein the reference sample is obtained from the same tissue as the test sample.

13. The method of claim 1, wherein the subject is a human.

14. The method of claim 1, further comprising treating the subject with a chemotherapeutic agent, radiation therapy, cryotherapy, hormone therapy, or immunotherapy.

15. The method of claim 14, wherein the chemotherapeutic agent comprises dacarbazine, temozolomide, nab-paclitaxel, paclitaxel, cisplatin, or carboplatin.

16. The method of claim 4, further comprising administering an inhibitor of the CD47 gene, thereby treating the leukemia.

17. The method of claim 16, wherein the inhibitor comprises a small molecule inhibitor, RNA interference (RNAi), an antibody, an antibody fragment, an antibody drug conjugate, an aptamer, a chimeric antigen receptor (CAR), a T cell receptor, or any combination thereof.

18. The method of claim 17, wherein the antibody or antibody fragment is partially humanized, fully humanized, or chimeric.

19. The method of claim 17, herein the antibody or antibody fragment comprises a nanobody, an Fab, an Fab′, an (Fab′)2, an Fv, a single-chain variable fragment (ScFv), a diabody, a triabody, a tetrabody, a Bis-scFv, a minibody, an Fab2, an Fab3 fragment, or any combination thereof.

20. The method of claim 1, further comprising administering to the subject an anti-CTLA4 antibody, thereby treating the leukemia.

21. The method of claim 1, wherein the CTLA4 antagonist is Ipilimumab.

22. A composition for predicting no clinical benefit in response to CTLA4 therapy comprising a CD47 molecule (CD47) gene synthesized complementary deoxyribonucleic acid (cDNA).

23. The composition of claim 22, wherein the CD47 gene is immobilized on a solid support.

24. The composition of claim 23, wherein the CD47 gene is linked to a detectable label.

25. The composition of claim 24, wherein the detectable label comprises a fluorescent label, a luminescent label, a chemiluminescent label, a radiolabel, a SYBR Green label, or a Cy3-label.

26. (canceled)

27. (canceled)

28. (canceled)

29. A method of treating cancer in a subject in need thereof, comprising:
identifying the subject as having aberrant expression of at least one resistant cancer-associated gene; and
co-administering a therapeutically effective amount of one or more CTLA4 inhibitor agents and one or more inhibitors of the at least one resistant cancer-associated genes to the subject, thereby treating the cancer; or
a method of treating cancer in a subject in need thereof, comprising:
administering a therapeutically effective amount of one or more CTLA4 inhibitor agents to the subject, wherein the subject is identified as not having aberrant expression of at least one resistant cancer-associated gene.

30. The method of claim 29, wherein the wherein the CTLA4 antagonist is Ipilimumab.

31. The method of claim 29, wherein the at least one resistant cancer-associated gene is a CD47 molecule (CD47) gene.

32. The method of claim 29, wherein the cancer is leukemia.

33. The method of claim 29, wherein the one or more inhibitors comprise a small molecule inhibitor, RNA interference (RNAi), an antibody, an antibody fragment, an antibody drug conjugate, an aptamer, a chimeric antigen receptor (CAR), a T cell receptor, or any combination thereof.

34. The method of claim 33, wherein the antibody or antibody fragment is partially humanized, fully humanized, or chimeric.

35. The method of claim 33, wherein the antibody or antibody fragment comprises a nanobody, an Fab, an Fab′, an (Fab′)2, an Fv, a single-chain variable fragment (ScFv), a diabody, a triabody, a tetrabody, a Bis-scFv, a minibody, an Fab2, an Fab3 fragment, or any combination thereof.

36. The method of claim 29, further comprising administering to the subject an anti-CTLA4 antibody, thereby treating the leukemia.

37.

38. (canceled)

39. (Canceled)
